(19) United States
(12) Patent Application Publication
(10) Pub. No.: US 2004/0024202 A1
(43) Pub. Date: Feb. 5, 2004

(54) C-17 spirolactonization and 6,7 oxidation of steroids

(75) Inventors: Paula C. Miller, St. Louis, MO (US); Mark J. Pozzo, Chesterfield, MO (US); Shine K. Chou, Chesterfield, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/392,857

(22) Filed: Mar. 21, 2003

Related U.S. Application Data
(60) Provisional application No. 60/425,596, filed on Nov. 12, 2002.
(60) Provisional application No. 60/411,874, filed on Sep. 19, 2002.
(60) Provisional application No. 60/366,784, filed on Mar. 22, 2002.

(51) Int. Cl. C07J 21/00

(52) U.S. Cl. 540/46

(57) ABSTRACT

Novel processes for the C-17 spirolactonization and 6,7 oxidation of steroid compounds are provided. In certain preferred embodiments, the present invention provides for the preparation of steroid compounds which are useful in the preparation of methyl hydrogen 9,11α-epoxy-17α-hydroxy-3-oxopregn-4-ene-7α,21-dicarboxylate, γ-lactone (otherwise referred to as eplerenone or epoxymexrenone).

### CROSS-REFERENCE TO RELATED APPLICATIONS

This application claims priority from U.S. Provisional Application Serial No. 60/425,596, filed Nov. 12, 2002, U.S. Provisional Application Serial No. 60/411,874, filed Sep. 19, 2002 and U.S. Provisional Application Serial No. 60/366,784, filed Mar. 22, 2002. The texts of U.S. Provisional Application Serial No. 60/425,596, U.S. Provisional Application Serial No. 60/411,874 and U.S. Provisional Application Serial No. 60/366,784 are hereby incorporated herein by reference in their entirety.

### BACKGROUND OF THE INVENTION

This invention generally relates to processes for preparing steroid compounds, and more particularly, to processes for the C-17 spirolactonization and 6,7 oxidation of steroid compounds. Most particularly, the invention relates to processes for the C-17 spirolactonization and 6,7 oxidation of steroid compounds which are useful in the preparation of methyl hydrogen 9,11α-epoxy-17α-hydroxy-3-oxopregn-4-ene-7α,21-dicarboxylate, γ-lactone (otherwise referred to as eplerenone or epoxymexrenone).

Methods for the preparation of 9,11 epoxy steroids in general, and eplerenone in particular, are described in International Publication WO 98/25948 and U.S. Pat. No. 6,331,622, U.S. Pat. No. 6,180,780 and U.S. Pat. No. 5,981,744, the entire texts of which are hereby incorporated herein by reference. Further methods for preparing 9,11 epoxy steroids, and eplerenone in particular, are described in co-assigned U.S. patent application Ser. No. ______, entitled “Processes To Prepare Eplerenone”, which was filed on even date herewith and the text of which is hereby incorporated herein by reference in its entirety.

### SUMMARY OF THE INVENTION

This invention provides for, in part, improved processes for the C-17 spirolactonization and 6,7 oxidation of steroid compounds. Among the objects of certain preferred embodiments of the invention are the provision of such a process wherein high purity spirolactone steroid compounds are produced in high yield; the provision of such a process wherein a broad range of substrates may be used; and the provision of such a process which may be implemented with reasonable capital expense and operated at reasonable conversion cost.

Accordingly, in a first embodiment, the present invention is directed to a process for the preparation of a steroid compound corresponding the Formula II:

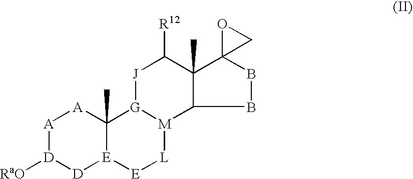

wherein:

R^{a} is alkyl;

R¹² is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

B-B represents the group —CHR¹⁵—CHR¹⁶— or an α-oriented or β-oriented cyclic group:

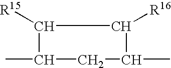

where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

G-J represents the group:

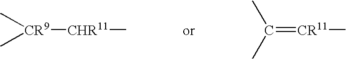

where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

D-D represents the group:

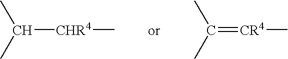

where R⁴ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

E-E represents the group:

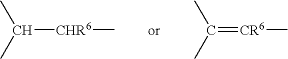

where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and

L-M represents the group:

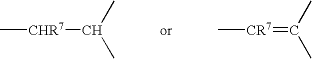

where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl.

The process comprises contacting a steroid substrate with a base and a solvent medium containing a sulfonium salt to produce a product mixture comprising the compound of Formula II above, wherein the steroid substrate comprises a compound corresponding to Formula I:

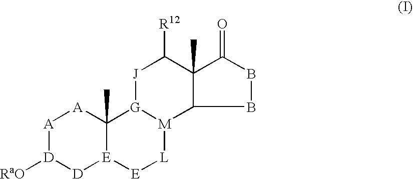

wherein the substituents R^{a}, R¹², A-A, B-B, D-D, E-E, G-J and L-M are as defined in Formula II above.

In another embodiment, the invention is directed to a process for the preparation of a steroid compound corresponding to the Formula III:

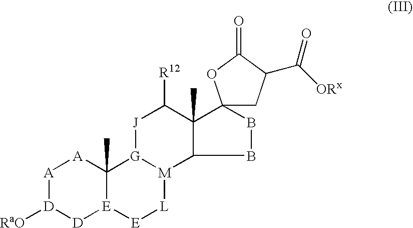

wherein:

R^{a} and R^{x} are independently selected from the group consisting of alkyl;

R¹² is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

B-B represents the group —CHR¹⁵—CHR¹⁶— or an α-oriented or β-oriented cyclic group:

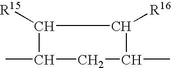

where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

G-J represents the group:

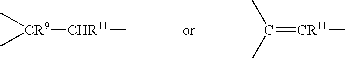

where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

D-D represents the group:

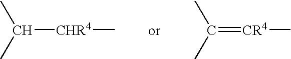

where R⁴ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

E-E represents the group:

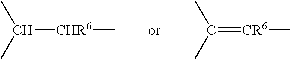

where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and

L-M represents the group:

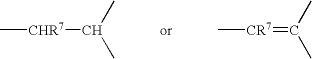

where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl.

The process comprises contacting a steroid substrate with a malonic acid diester and a base in the presence of a solvent to produce a product mixture comprising the compound of Formula III, wherein the steroid substrate comprises a compound corresponding to the Formula II above. The process is further characterized in that the product mixture is treated to remove or sequester base.

In another embodiment, the invention is directed to a process for-preparing a steroid compound of Formula III as described immediately above. The process comprises contacting a steroid substrate of Formula II described above with diethyl malonate and sodium ethoxide to produce a product mixture comprising the steroid compound corresponding to Formula III.

In still another embodiment, the invention is further directed to a process for preparing a steroid compound corresponding to the Formula VI:

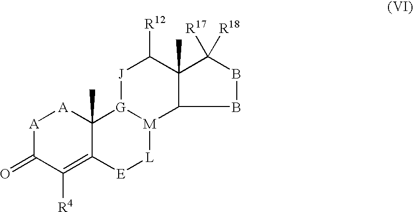

wherein:

R⁴ and R¹² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

R¹⁷ and R¹⁸ are independently selected from the group consisting of hydrogen, alkyl, hydroxy, alkenyl and alkynyl; or R¹⁷ and R¹⁸ together form a ketal or keto group; or R¹⁷ and R¹⁸ together with the C₁₇ carbon to which they are attached form the α-oriented or β-oriented cyclic structure:

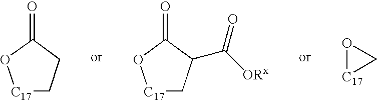

where Rx is alkyl;

A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

B-B represents the group —CHR¹⁵—CHR¹⁶— or an α-oriented or β-oriented cyclic group:

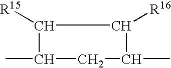

where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

G-J represents the group:

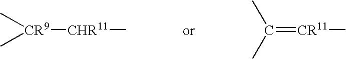

where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

E-L represents the group —CHR⁶—CHR⁷— or —CR⁶═CR⁷—, where R⁶ and R⁷ are independent, R⁶ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and R⁷ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl; and

M-G represents the group:

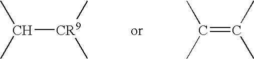

where R⁹ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy.

The process comprises oxidizing an enol ether steroid substrate corresponding to Formula V to produce a product mixture comprising the steroid compound of Formula VI, wherein the enol ether steroid substrate corresponds to a compound of Formula V:

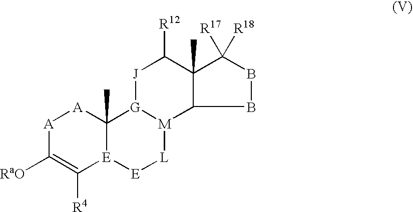

wherein:

R^{a} is alkyl;

E-E represents the group:

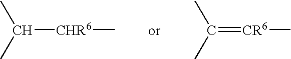

where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

L-M represents the group:

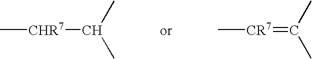

where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl; and

the substituents R⁴, R², R⁷, R¹⁸, A-A, B-B and G-J are as defined above with respect to Formula VI.

In another embodiment, the invention is directed to a process for the preparation of a steroid compound corresponding to Formula VI as described above. The process comprises contacting a steroid substrate corresponding to Formula V as described above with an oxidizing agent in the presence of water to produce a product mixture comprising a steroid compound corresponding to Formula VI. The steroid compound corresponding to Formula VI is then recovered from the product mixture by contacting the product mixture with a base.

In another embodiment, the invention is directed to a process for the preparation of a steroid compound corresponding to Formula VI-A:

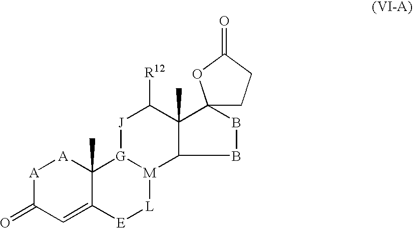

wherein:

R¹² is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

B-B represents the group —CHR¹⁵—CHR¹⁶— or an α-oriented or β-oriented cyclic group:

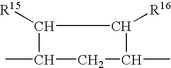

where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

G-J represents the group:

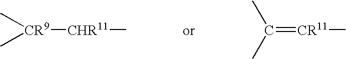

where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

E-L represents the group —CHR⁶—CHR⁷— or —CR⁶═CR⁷—, where R⁶ and R⁷ are independent, R⁶ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and R⁷ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl; and

M-G represents the group:

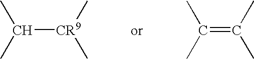

where R⁹ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy.

The process comprises contacting a steroid substrate corresponding to a compound of Formula I:

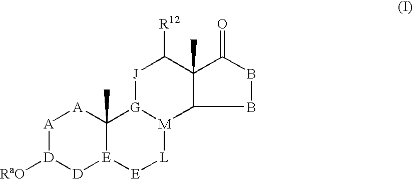

wherein:

R^{a} is alkyl;

D-D represents the group:

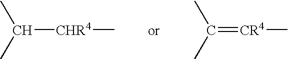

where R⁴ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

E-E represents the group:

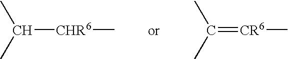

where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

L-M represents the group:

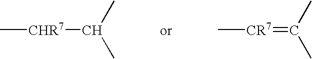

where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl; and

the substituents R¹², A-A, B-B and G-J are as defined in Formula VI-A,

with a base and a solvent medium containing a sulfonium salt to produce an oxirane intermediate steroid compound of Formula II:

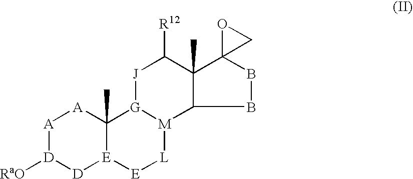

wherein the substituents R^{a}, R¹², A-A, B-B, G-J, D-D, E-E and L-M are as defined in Formula I. The oxirane intermediate compound of Formula II is then contacted with a malonic acid diester and a base in the presence of a solvent to produce a dicarboxylate intermediate steroid compound corresponding to Formula III:

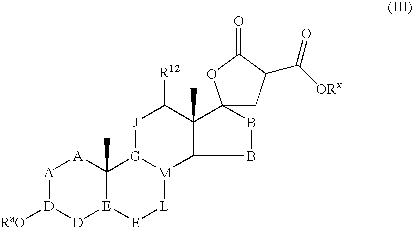

wherein R^{x} is alkyl and the substituents R^{a}, R¹², A-A, B-B, G-J, D-D, E-E and L-M are as defined in Formula I. The process further comprises decarboxylating the dicarboxylate intermediate compound of Formula III to produce an enol ether steroid compound of Formula V-A:

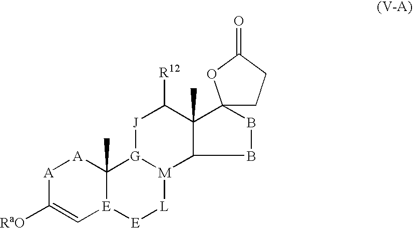

wherein the substituents R^{a}, R¹² A-A, B-B, G-J, E-E and L-M are as defined in Formula I and oxidizing the enol ether steroid compound of Formula V-A to produce the steroid compound of Formula VI-A.

In another embodiment, the invention is directed to a process for the preparation of a steroid compound corresponding to Formula VI-C:

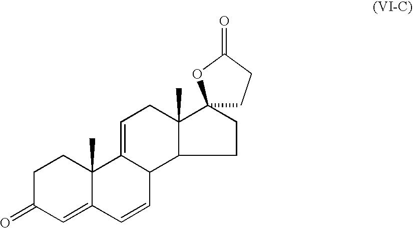

The process comprises contacting a steroid substrate corresponding to Formula I-A:

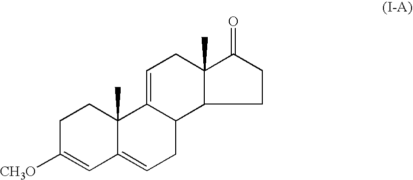

with a base and a solvent medium containing a sulfonium salt to produce an oxirane intermediate compound of Formula II-C:

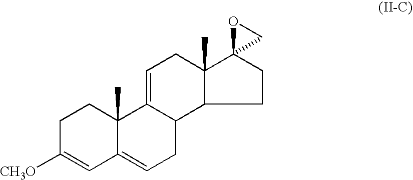

The oxirane intermediate compound of Formula II-C is then contacted with a malonic acid diester and a base in the presence of a solvent to produce a dicarboxylate intermediate steroid compound corresponding to Formula III-C:

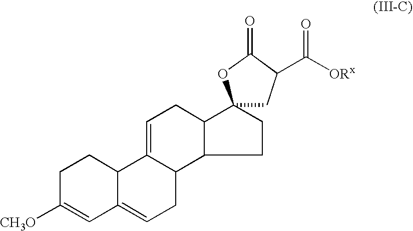

The process further comprises decarboxylating the dicarboxylate intermediate compound of Formula III-C to produce an enol ether steroid compound of Formula IV-C:

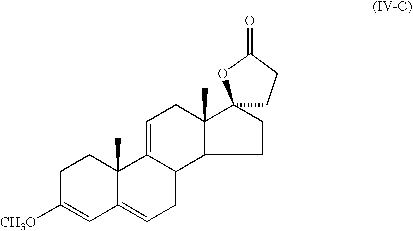

and oxidizing the enol ether steroid compound of Formula IV-C to produce the steroid compound of Formula VI-C.

In another embodiment, the invention is further directed to a process for preparing a steroid substrate corresponding to Formula X:

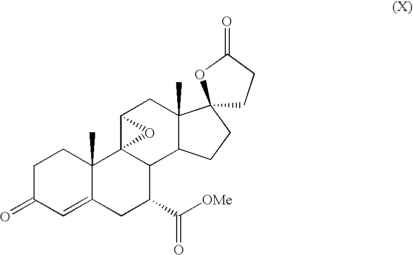

The process comprises contacting a steroid substrate of Formula I-A with a base and a solvent medium comprising a sulfonium salt to produce an oxirane intermediate steroid compound of Formula II-C:

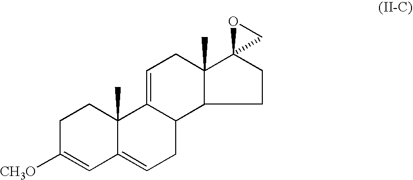

The steroid substrate of Formula I-A corresponds to the compound:

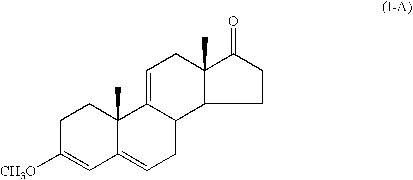

The process further comprises contacting the oxirane intermediate steroid compound of Formula II-C with a malonic acid diester and a base in the presence of a solvent to produce a dicarboxylate intermediate steroid compound of Formula III-C:

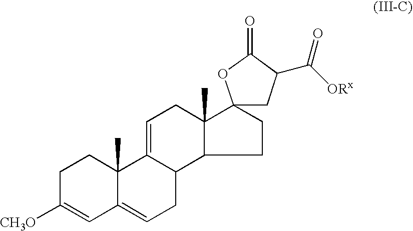

The dicarboxylate intermediate compound of Formula III-C is contacted with an alkali metal halide and water in the presence of a solvent to produce an enol ether steroid compound of Formula IV-C:

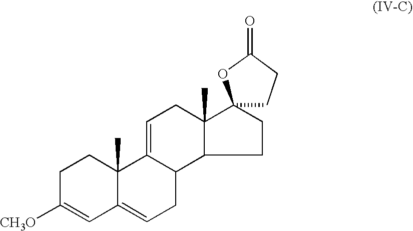

The enol ether steroid compound of Formula IV-C is then oxidized to form a dienone steroid compound corresponding to Formula VI-C:

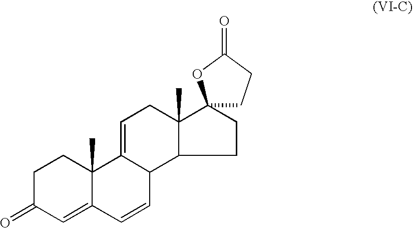

The dienone steroid compound of Formula VI-C is then contacted with an alkyl furan and a Lewis acid to produce a 7α-furyl intermediate compound of Formula VII:

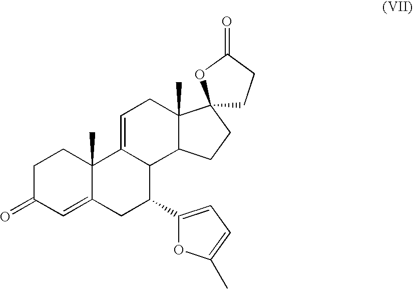

The 7α-furyl intermediate compound of Formula VII is subsequently converted to a 7α-methoxycarbonyl intermediate compound of Formula IX:

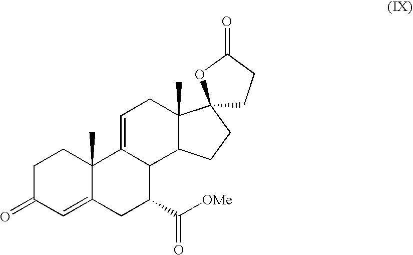

and the 7α-methoxycarbonyl intermediate compound of Formula IX is converted to the steroid product of Formula X.

In still another embodiment, the present invention is further directed to a steroid compound corresponding to Formula X:

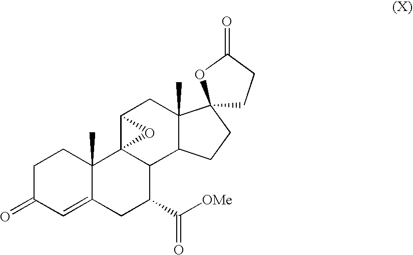

The steroid compound is characterized in that it is prepared by a process comprising contacting a steroid substrate of Formula I-A shown above with a base and a solvent medium comprising a sulfonium salt to produce an oxirane intermediate steroid compound of Formula II-C shown above. The process further comprises contacting the oxirane intermediate steroid compound of Formula II-C with a malonic acid diester and a base in the presence of a solvent to produce a dicarboxylate intermediate steroid compound of Formula III-C shown above. The dicarboxylate intermediate compound of Formula III-C is then contacted with an alkali metal halide and water in the presence of a solvent to produce an enol ether steroid compound of Formula IV-C shown above. The process further comprises oxidizing the enol ether steroid compound of Formula IV-C to form a dienone steroid compound corresponding to Formula VI-C shown above. The dienone steroid compound of Formula VI-C is then contacted with an alkyl furan and a Lewis acid to produce a 7α-furyl intermediate compound of Formula VII shown above. The 7α-furyl intermediate compound of Formula VII is then converted to a 7α-methoxycarbonyl intermediate compound of Formula IX shown above, which is finally converted to the steroid compound of Formula X.

Other objects of the invention will be in part apparent and in part pointed out hereinafter.

### DETAILED DESCRIPTION OF THE PREFERRED EMBODIMENTS

In accordance with the present invention, Applicants have discovered a process for the preparation of epoxy steroids having a spirolactone moiety at the 17-position. The process of the present invention generally comprises four steps including an oxirane formation, a malonate condensation, a decarboxylation and an oxidation. In particular, as is demonstrated below, an embodiment of the present invention provides a novel process for the preparation of eplerenone (methyl hydrogen 9,11α-epoxy-17α-hydroxy-3-oxopregn-4-ene-7α,21-dicarboxylate, γ-lactone, and sometimes otherwise referred to as epoxymexrenone).

Steroid Substrate

The beginning substrate for the process of the present invention generally comprises a steroid compound corresponding to the Formula I:

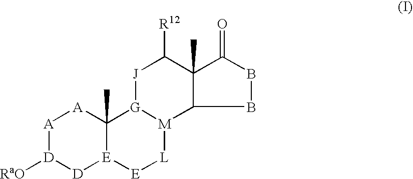

wherein:

R^{a} is alkyl;

R¹² is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

B-B represents the group —CHR¹⁵—CHR¹⁶— or an α-oriented or β-oriented cyclic group:

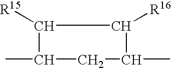

where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

G-J represents the group:

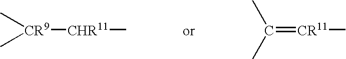

where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

D-D represents the group:

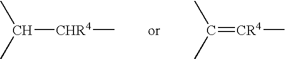

where R⁴ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

E-E represents the group:

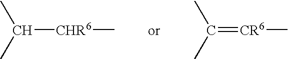

where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and

L-M represents the group:

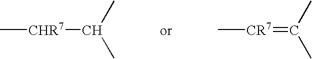

where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl, wherein the furyl or substituted furyl substituent is selected from the group consisting of a molecular fragment of the formula (-A1)

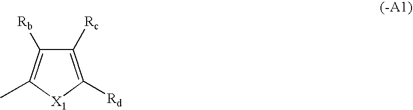

or of the formula (-A2)

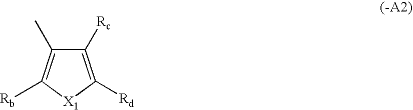

where

X₁ is —S—, —O— or —NX₁₋₁— and

where X₁₋₁ is —H, C₁-C₄ alkyl, —CO—OX₁₋₂ where X₁₋₂ is C₁-C₄ alkyl or —CH₂-Ø, —CO—X₁₋₂ where X₁₋₂ is as defined above, —CO-Ø where -Ø is substituted in the o-position with —CO—O—(C₁-C₄ alkyl), —SO₂—(C₁-C₃ alkyl), —SO₂-Ø where Ø is optionally substituted with 1 or 2 C₁-C₄ alkyl, C₁-C₄ alkoxy;

R_{b} is selected from the group consisting of —H, C₁-C₄ alkyl and phenyl optionally substituted with 1 or 2 C₁-C₄ alkyl, C₁-C₄ alkoxy;

R_{c} is selected from the group consisting of —H, C₁-C₄ alkyl, C₁-C₄ alkoxy, —O—Si(R)₃ (where the each R is independently selected from the group consisting of —H, C₁-C₄ alkyl, -Ø, C₁-C₄ alkoxy and —OH, —F, —Cl, —Br, —I, —CO—OCH₃) and —CO—R_{b-1} (where R_{b-1} is C₁-C₄ alkyl or -Ø);

R_{d} is selected from the group consisting of —H, —C≡N, C₁-C₁₀ alkyl, C₁-C₄ alkoxy, —CH₂—OR_{d-1} (where R_{d-1} is —H or C₁-C₄ alkyl), —CH₂—N(R_{d6})₂ (where the two R_{d-6} are independently selected from the group consisting of C₁-C₄ alkyl and -Ø), —CO—R_{d-6a} (where R_{d-6a} is C₁-C₄ alkyl or -Ø), —CH₂—O—CO—R_{d-1} (where R_{d-1} is as defined above), —CH(OR_{d-1})₂ (where R_{d-1} is as defined above and where the two R_{d-1} taken together are —CH₂—CH₂—, —CH₂—CH₂—CH₂—, —CH₂—C(CH₃—)₂—CH₂—), —CH(—O—CO—R_{d-1})₂ (where R_{d-1} is as defined above), —Si(R)₃ (where R is as defined above), —O—Si(R)₃ (where R is as defined above), —Sn(R_{b1})₃ (where R_{b1} is as defined above), —S—R_{d-5} (where R_{d-5} is C₁-C₄ alkyl or -Ø), and —N(R_{d-6})₂ (where R_{d-6} is as defined above);

R_{c} and R_{d} taken together with the atoms to which they are attached form

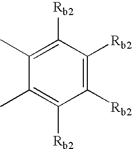

where the R_{b2} are the same or different and are as defined above,

—CR_{b2}═M (-B)

where R_{b2} is selected from the group consisting of —H, C₁-C₄ alkyl, —F, —Cl, —Br, —I, —OR_{b2-1} where R_{b2-1} is —H, C₁-C₄ alkyl, -Ø or —SiR_{b2-2}R_{b2-3}R_{b2-4} where R_{b2-2}, R_{b2-3} and R_{b2-4} are the same or different and are C₁-C₄ alkyl or C₁-C₄ alkoxy; —S—R_{b2-5} where R_{b2-5} is C₁-C₄ alkyl or -Ø; —S—(O)₁₋₂—R_{b2-5} where R_{b2-5} is as defined above; —N (R_{d6})₂ where the two R_{d6} are the same or different and are as defined above; —P(O)(O—R_{b2-1})₂ where R_{b2-1} is as defined above; —Si(R)₃ where R is as defined above;

where M is:

(1) ═O;

(2) ═N—R_{c2} where R_{c2} is selected from the group consisting of —H, C₁-C₄ alkyl, C₁-C₄ alkenyl containing 1 or 2 double bonds, C₁-C₄ alkynyl containing 1 triple bond, —CO—OR_{c2-6} where R_{c2-6} is —H or C₁-C₄ alkyl, —C(R_{c2-6})₂—OR_{c2-1} where R_{c2-6} are the same or different and are as defined above and where R_{c2-1} is C₁-C₄ alkyl, -Ø or —Si(R)₃ where the three R are the same or different and are defined above, —OR_{c2-1} where R_{c2-1} is as defined above, —S—R_{c2-5} where R_{c2-5} is C₁-C₄ alkyl or -Ø, —S—(O)₁₋₂—R_{c2-5} where R_{c2-5} is as defined above, —N(R_{d6})₂ where the two R_{d6} are the same or different and are as defined above, and —Si(R)₃ where the three R are as defined above;

(3) ═C(R_{c2})₂ where R_{c2} are the same or different and are as defined above;

where R_{b2} and R_{c2} are taken together with the atoms to which they are attached to form a ring of 5 thru 7 members, optionally containing 3 thru 5—O—, —S—, —N═, —NX₁₋₁— where X₁₋₁ is as defined above, —CR_{b2}═ where R_{b2} is as defined above, —C(R_{b})₂— where R_{b} is as defined above, and optionally containing 1 or 2 additional double bonds;

—C═C—R_{c2} (-C)

where R_{c2} is as defined above;

—CH₂—CH═CH₂ (-D1)

—CH═C═CH₂ (-D2)

—CH₂—C≡C—H (-D3)

In a particularly preferred embodiment, the beginning steroid substrate is a compound corresponding to the Formula I-A:

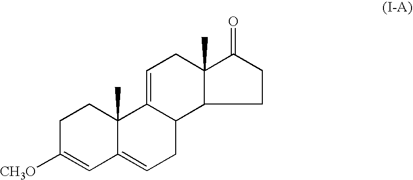

Step 1: Oxirane Formation

In a first embodiment, the process of the present invention is directed to the formation of an oxirane substituent at the C-17 position on a steroid substrate corresponding to a compound of Formula I defined above. The oxirane formation reaction generally comprises contacting the steroid substrate with a base and a solvent medium comprising a sulfonium salt to form a product mixture comprising an oxirane intermediate product corresponding to a compound of Formula II:

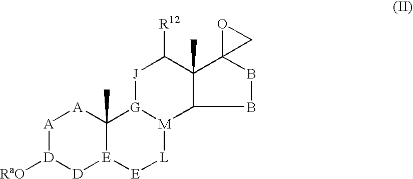

wherein the substituents R^{a}, R¹², A-A, B-B, G-J, D-D, E-E, and L-M are as defined in Formula I above. The oxirane formation reaction is summarized as shown in the following Reaction Scheme A:

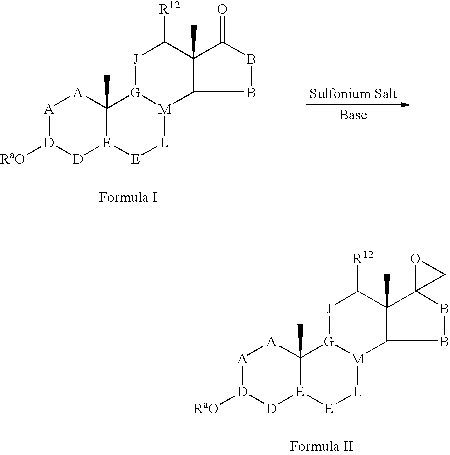

In a particularly preferred embodiment, the oxirane formation reaction comprises contacting a steroid substrate corresponding to the compound of Formula I-A described above with a base and a solvent medium containing a sulfonium salt to prepare an oxirane intermediate product comprising a compound of Formula II-A, as shown in Reaction Scheme Al:

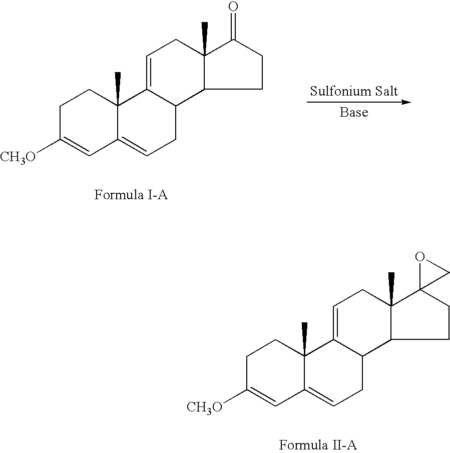

In accordance with the process of the present invention, Applicants have discovered that the oxirane formation reaction is effective in preparing an oxirane intermediate product mixture wherein the Reoriented oxirane compound of Formula II is formed in preference to the α-oriented oxirane compound of Formula II. As used herein, the β-oriented oxirane compound of Formula II corresponds to a compound of Formula II-B:

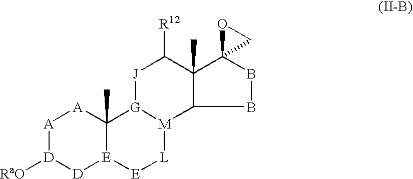

wherein the substituents R^{a}, R¹², A-A, B-B, G-J, D-D, E-E, and L-M are as defined in Formula I above. For example, it has been found that the reaction conditions, the base and the solvent medium containing the sulfonium salt can be selected as described herein to yield an oxirane intermediate product having a ratio of β-oriented oxirane compound to α-oriented oxirane compound of at least about 70:30 (β-oxirane/α-oxirane), more preferably a ratio of at least about 90:10 (β-oxirane/α-oxirane), and even more preferably a ratio of at least about 95:5 (β-oxirane/α-oxirane).

Thus, in the certain preferred embodiment shown above in Reaction Scheme A1, the reaction conditions and reactants of the oxirane formation reaction are preferably selected such that the product mixture comprises an oxirane intermediate steroid compound corresponding to Formula II-C:

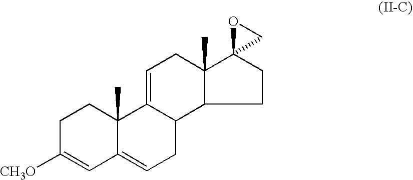

Suitable sulfonium salts for use in the oxirane formation reaction comprise trialkylsulfonium salts, particularly trimethylsulfonium salts, with trimethylsulfonium methyl sulfate being particularly preferred. Suitable solvents for use as the solvent medium of the sulfonium salt include dimethylsulfoxide, diethyl ether, dioxanes, diglyme, triglyme, dimethylformamide, tetrahydrofuran, dimethylacetamide, acetonitrile and mixtures thereof. Preferably the solvent medium for the sulfonium salt comprises dimethylsulfoxide, tetrahydrofuran or mixtures thereof.

The molar ratio of sulfonium salt to base charged to the reaction is from about 0.75:1 to about 1.5:1 (sulfonium salt/base), more preferably from about 0.9:1 to about 1.1:1 (sulfonium salt/base). Suitable bases for use in the oxirane formation reaction comprise alkali metal hydroxides, alkali metal hydrides, t-butyl alkali metal alkoxides and alkaline earth metal hydroxides. Preferred alkali metal hydroxide bases include potassium hydroxide, sodium hydroxide, lithium hydroxide and mixtures thereof, particularly in the form of a solid particulate. Preferred alkali metal hydride bases include potassium hydride, sodium hydride, lithium hydride and mixtures thereof. Preferred t-butyl alkali metal alkoxide bases include potassium t-butoxide, sodium t-butoxide, lithium t-butoxide and mixtures thereof. For example, in certain preferred embodiments described below, the base comprises potassium hydroxide or potassium t-butoxide.

Although the oxirane formation reaction is generally viable by contacting the steroid substrate with the base and a sulfonium salt in any order, certain preferred embodiments of the present invention are directed to contacting the steroid substrate with a solvent medium containing the sulfonium salt. Without being held to a particular theory, it is believed that contacting the steroid substrate with a solvent medium containing the sulfonium salt may be important in producing favorable results concerning product yield and/or β-oxirane/α-oxirane ratios as described above. Further, use of the sulfonium salt in a solvent medium is advantageous in commercial practice of the present invention because particulate solid sulfonium salts are hygroscopic in nature and generally difficult to handle.

Thus, in one preferred embodiment, the process of the present invention comprises preparing a substrate pre-mixture comprising the steroid substrate and the base in a solvent medium and contacting the substrate pre-mixture with the solvent medium containing the sulfonium salt. Suitable solvents for use as the solvent medium of the substrate pre-mixture include those of the solvent medium for the sulfonium salt described above such as solvents selected from the group consisting of dimethylsulfoxide, diethyl ether, dioxanes, diglyme, triglyme, dimethylformamide, tetrahydrofuran, dimethylacetamide, acetonitrile and mixtures thereof. It is important to note that the solvent selected as the solvent medium of the substrate pre-mixture and the solvent selected as the solvent medium containing the sulfonium salt may be the same solvent or may comprise different solvents. For example, in a preferred embodiment further described below, the invention employs tetrahydrofuran as the solvent of the substrate pre-mixture and dimethylsulfoxide as the solvent medium containing the sulfonium salt. Alternatively, other preferred embodiments described herein employ tetrahydrofuran as the solvent medium for both the substrate pre-mixture and the solvent medium containing the sulfonium salt.

When the process involves forming a substrate pre-mixture comprising the steroid substrate and the base prior to contact with the solvent medium containing the sulfonium salt, it has been found that controlling the temperature of the steroid pre-mixture prior to contact with the solvent medium containing the sulfonium salt may be beneficial in preventing substrate degradation. Thus, in a preferred embodiment, it is desirable to maintain the temperature of the substrate pre-mixture at or below about 15° C., more preferably at or below a temperature of about 10° C., and even more preferably at about 5° C. prior to the addition of the solvent medium containing the sulfonium salt. After the solvent medium containing the sulfonium salt is contacted with the substrate pre-mixture, the temperature of the reaction may be allowed to proceed to reflux, which typically occurs from about 75° to about 85° C., with a reaction temperature of about 65° C. preferred.

In a first preferred embodiment, the oxirane formation reaction comprises preparing a substrate pre-mixture by combining the steroid substrate and solid particulate potassium hydroxide as the base in tetrahydrofuran as a solvent. The substrate pre-mixture is then contacted with a solvent medium comprising dimethylsulfoxide containing trimethylsulfonium methylsulfate as the sulfonium salt. An example of such an embodiment of the present invention is demonstrated in Example 3 herein.

Alternatively, another preferred embodiment of the present invention comprises preparing a substrate pre-mixture by contacting the steroid substrate with the solvent medium comprising tetrahydrofuran and further containing trimethylsulfonium methylsulfate as the sulfonium salt. The substrate pre-mixture is then contacted with potassium t-butoxide as the base to complete the oxirane formation reaction. An example of such an embodiment wherein the steroid substrate comprises 3-methoxy-androsta-3,5,9(11)-trien-17-one is demonstrated in Example 2 herein.

It is important to note that in accordance with the present invention, Applicants have advantageously discovered that the each of the embodiments of the process of the invention described above may further comprise preparing the sulfonium salt in situ. In particular, it has been found that the sulfonium salt may be prepared by contacting dimethyl sulfide with an alkylating agent in the presence of a solvent medium. Typically, the alkylating agent comprises dimethylsulfate or methyl iodide, with dimethylsulfate being particularly preferred. For example, contacting methyl sulfate with dimethyl sulfate in the presence of a solvent medium produces a solvent medium containing a trimethylsulfonium salt comprising trimethylsulfonium methyl sulfate, which may then be used directly in the oxirane formation reaction of the present invention.

After the oxirane formation reaction is complete, the product mixture is preferably cooled before recovering and isolating the oxirane intermediate steroid product. In embodiments of the present invention wherein the sulfonium salt comprises a trimethylsulfonium salt, experience to date suggests that the oxirane formation product mixture may further comprise by-product dimethylsulfide. Thus, in certain preferred embodiments, it may be desirable to further work up the product mixture to remove by-product dimethyl sulfide before recovering the oxirane intermediate steroid product. For example, in one embodiment, it has been found that by-product dimethyl sulfide can be removed from the product mixture by distillation to remove about one-third to about one-half of the initial solvent volume of the product mixture. Such distillation has generally been found to be sufficient to remove a significant portion of the dimethyl sulfide by-product from the product mixture.

After distillation, the oxirane intermediate steroid product may be recovered from the product mixture, preferably by precipitation. For example, in one embodiment, the product mixture is contacted with water to precipitate a solid product comprising the oxirane intermediate compound. Preferably, the precipitation comprises contacting the product mixture with about one to about five volumes of water over a period of less than about 30 minutes to recover a solid steroid product.

In a further embodiment, the process of the present invention comprises washing the recovered solid product containing the oxirane intermediate compound. Preferably, the solid product is washed by contacting the product with water. Even more preferably, the solid product is washed by contacting the solid product with water at a temperature of at least about 25° C., preferably at a temperature of from about 25° to about 60° C., and more preferably at a temperature of at least about 40° C.

In another preferred embodiment, the recovered solid product may be further washed by contacting the product with water followed by an alcohol. Suitable alcohols for washing the recovered solid product include methanol, ethanol, isopropanol, and t-butanol, with methanol being preferred. Wherein the water wash is preferably conducted at a temperature described above, the alcohol wash is preferably conducted at a temperature of from about 15° C. to about 30° C., preferably at a temperature of about 20° C.

After the recovered solid product has been washed, the product is preferably dried by contacting the solid product with air or nitrogen. In a particularly preferred embodiment, the recovered solid product is dried by contacting the product with nitrogen at a temperature of from about 20° to about 80° C., more preferably at a temperature of from about 60° to about 75° C., even more preferably at a temperature of about 70° C.

Step 2: Malonate Condensation

The second step of the process of the present invention comprises the malonate condensation of an oxirane steroid compound, particularly an oxirane intermediate steroid compound as described above and shown in Formula II wherein the substituents R^{a}, R¹², A-A, B-B, G-J, D-D, E-E, and L-M are as defined in Formula I above. The malonate condensation reaction generally comprises reacting an oxirane intermediate compound of Formula II with a malonic acid diester, a base and a solvent to form a product mixture comprising a dicarboxylate intermediate corresponding to the Formula III:

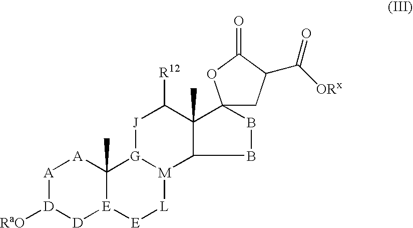

wherein R^{x} is alkyl; and the substituents R^{a}, R¹², A-A, B-B, G-J, D-D, E-E and L-M are as defined in Formula I. The process produces a product mixture comprising the dicarboxylate intermediate steroid compound of Formula III. In a particularly preferred embodiment, the process further comprises treating the product mixture to remove or sequester base.

The malonate condensation reaction is summarized as shown in the following Reaction Scheme B:

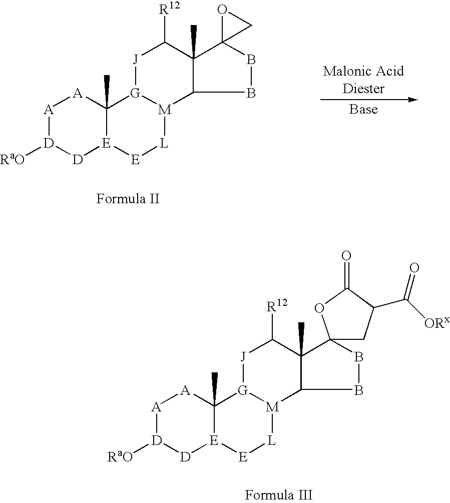

Preferably, the conditions of the malonate condensation reaction as described herein are selected such that the process summarized above in Reaction Scheme B produces a product mixture comprising the dicarboxylate intermediate steroid compound corresponding to Formula III-B:

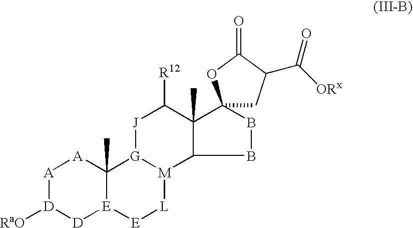

In a preferred embodiment, the malonate condensation reaction comprises reacting an oxirane intermediate compound of Formula II-A as defined above in Step 1 with a malonic acid diester and a base in the presence of a solvent to produce a product mixture comprising a dicarboxylate intermediate compound corresponding to Formula III-A, wherein R^{x} is alkyl. Such a preferred embodiment is shown in Reaction Scheme B1:

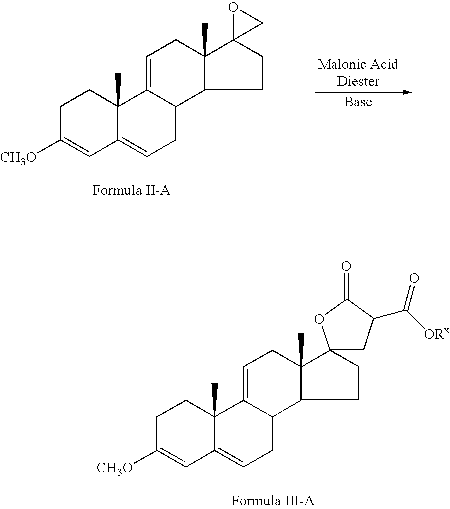

An even more preferred embodiment of the malonate condensation process as described above and shown in Reaction Scheme B1 produces a product mixture comprising the β-oriented dicarboxylate intermediate steroid compound of Formula III-A. As described herein, the β-oriented dicarboxylate intermediate steroid compound of Formula III-A corresponds to the Formula III-C:

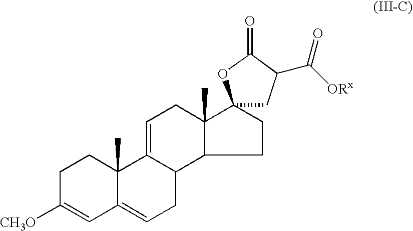

Preferred malonic acid diesters for use in the process of the present invention include alkyl malonates, particularly dimethyl malonate or diethyl malonate. Preferred bases for use in the process of the invention comprise alkali metal alkoxides, preferably sodium methoxide or sodium ethoxide. In a particularly preferred embodiment, the malonic acid diester comprises diethyl malonate and the base comprises sodium ethoxide.

It is important to note that the malonate condensation reaction should be conducted in an anhydrous environment. Therefore, the reaction is preferably conducted in the presence of an anhydrous solvent, preferably a solvent selected from the group consisting of an anhydrous alcohol, dimethylformamide, dimethylsulfoxide, dimethylacetamide and mixtures thereof. In a particularly preferred embodiment, the solvent comprises an anhydrous alcohol, most preferably anhydrous ethanol.

Although the malonate condensation reaction is generally viable by contacting the reactants in any order, Applicants have discovered certain orders of addition which may be preferred in some applications of the present invention. For example, in accordance with the present invention, Applicants have discovered that certain orders of adding the reactants into the malonate condensation reaction may lead to increased yield of the dicarboxylate intermediate steroid product and/or less product impurities. Therefore, in certain preferred embodiments, the process of the present invention comprises preparing a steroid substrate pre-mixture comprising the steroid substrate, the malonic acid diester and a solvent. The malonate condensation reaction is then commenced by contacting the steroid substrate pre-mixture and the base to prepare the product mixture comprising the dicarboxylate intermediate steroid product. In an alternative embodiment, the process may comprise preparing a malonate pre-mixture comprising the base, the malonic acid diester and a solvent. The malonate pre-mixture is then contacted with the steroid substrate to produce the product mixture comprising the dicarboxylate intermediate steroid compound.

In other preferred embodiments, the product mixture is treated to remove or sequester base after the condensation reaction is complete. For example, without being held to a particular theory, Applicants have discovered that treating the product mixture to remove or sequester base may avoid unnecessary degradation of the steroid product within the product mixture under basic conditions, which thereby allows for increased product yields. While not necessary or critical to the present invention, the product mixture may alternatively be cooled prior to being treated to remove or sequester base. For example, it has been found that the product mixture may be cooled to a temperature of about 40° C. before base is removed or sequestered. Preferably, the product mixture is treated at a temperature of from about 40° to about 75° C.

The product mixture may be treated by any means generally known in the art which are suitable for removing or sequestering base within a liquid medium. For example, in a preferred embodiment, the product mixture is treated to remove base from the product mixture by neutralization. Typically, the product mixture may be treated by contacting the product mixture with an acid or an acid source, preferably an acid or acid source which is soluble in the reaction medium and which would be characterized by one skilled in the art as having a relatively low water content. It is important to note that the water content of the acid source should be limited to avoid hydrolytic attack on the steroid product during treatment of the product mixture. Examples of preferred acids for treating the product mixture include acids selected from the group consisting of acetic acid, formic acid, propionic acid, sulfuric acid, phosphoric acid, hydrochloric acid and mixtures thereof. In a particularly preferred embodiment, the product mixture is treated by contacting the product mixture with acetic acid. Other acids may be suitable for treating the product mixture including, for example, formic acid, propionic acid, concentrated sulfuric acid, 85% phosphoric acid and hydrochloric acid.

The proportion of acid to be contacted with the product mixture is typically from about 0.75 molar equivalents to about 1.5 molar equivalents of acid. For example, it has been found that proportions of acid below about 0.75 molar equivalents are insufficient while proportions greater than about 1.5 molar equivalents are not desired. In a preferred embodiment, about 0.85 to about 1.05 molar equivalents of acid are contacted with the product mixture as a treatment to remove or sequester base.

In other preferred embodiments of the invention, the dicarboxylate intermediate steroid product corresponding to Formula III is recovered from the product mixture after treating the product mixture to remove or sequester base. Preferably the dicarboxylate intermediate steroid product is recovered from the product mixture as a solid, most preferably by precipitation.

After precipitation, the recovered steroid product may preferably be washed. The recovered steroid product may be washed by contacting the product with water or an alcohol. Preferably, the recovered product is washed by contacting the product with a mixture of water and alcohol, more preferably an alcohol/water mixture comprising from about 25% to about 50% by weight alcohol, with an alcohol/water mixture comprising about 30% by weight alcohol being most preferred.

The recovered steroid product may be further dried by contacting the product with air or nitrogen. In a preferred embodiment, the recovered steroid product is dried by contacting the product with nitrogen at a temperature below about 100° C., preferably at a temperature of from about 20° C. to about 70° C., more preferably at a temperature of about 60° C.

Step 3: Decarboxylation

The third step of the process of the present invention generally comprises decarboxylating a dicarboxylate steroid compound of Formula III, preferably a dicarboxylate intermediate steroid compound such as that produced by Step 2 described above. Generally, the process comprises reacting a steroid substrate corresponding to Formula III as described above with an alkali metal halide in the presence of a solvent to form an enol ether steroid product corresponding to the Formula IV:

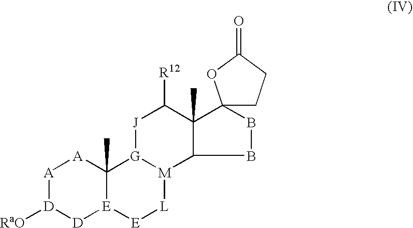

wherein the substituents R^{a}, R¹², A-A, B-B, G-J, D-D, E-E and L-M are as defined in Formula I.

The decarboxylation reaction is summarized as shown in Reaction Scheme C.

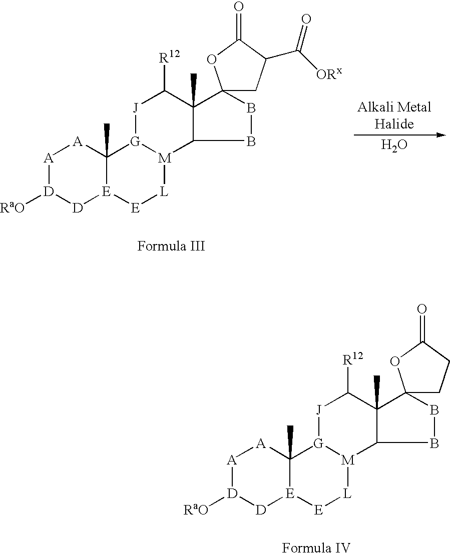

Preferably, the conditions of the decarboxylation reaction as described herein are selected such that the process summarized above in Reaction Scheme C produces a product mixture comprising the enol ether steroid compound corresponding to Formula IV-B:

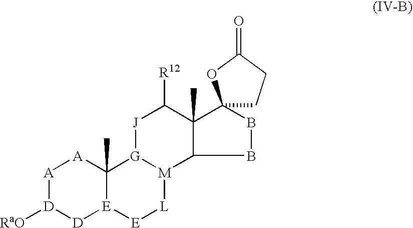

In a particularly preferred embodiment, the dicarboxylate intermediate comprises a compound corresponding to Formula III-A above and the enol ether steroid product comprises a compound corresponding to Formula IV-A, as shown in Reaction Scheme C1:

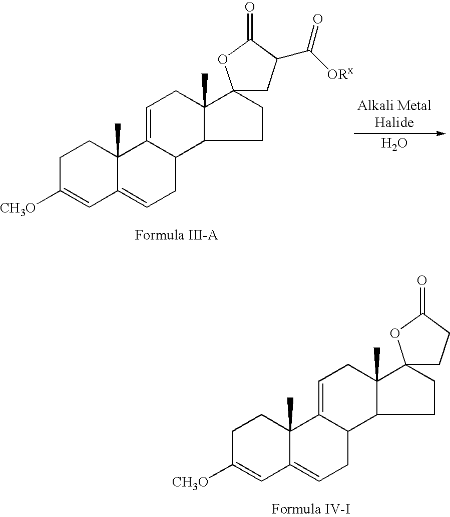

An even more preferred embodiment of the decarboxylation reaction as described above and shown in Reaction Scheme C1 produces a product mixture comprising the Reoriented enol ether steroid compound of Formula IV-A. As described herein, the Reoriented enol ether steroid compound of Formula IV-A corresponds to the Formula IV-C:

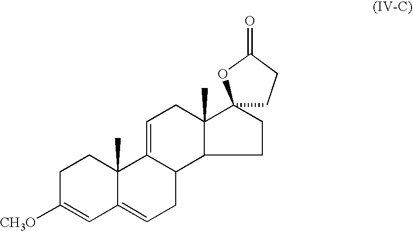

Generally, the decarboxylation reaction corresponds essentially to that described in U.S. Pat. Nos. 3,897,417, 3,413,288 and 3,300,489, which are expressly incorporated herein by reference. While the substrates of the present invention differ from those described in the '417, '288 and '489 patents, the reagents, mechanisms and conditions for introduction of the 17-spirolactone moiety are essentially the same. In a particularly preferred embodiment of the present invention, the alkali metal halide comprises sodium chloride and the solvent comprises dimethylformamide.

Typically, the reaction is heated to increase the rate of the reaction. Reaction temperatures up to reflux of the reaction system, depending upon the solvent used in the reaction, are generally acceptable. For example, in preferred embodiments wherein dimethylformamide is selected as the reaction solvent, the reaction may be heated to temperatures of from about 115° C. to about 150° C., with reaction temperatures of from about 130° to about 145° C. being preferred.

Step 4: Oxidation

In accordance with the process of the present invention, Applicants have discovered an improved process for the oxidation of steroid substrates generally corresponding to Formula V:

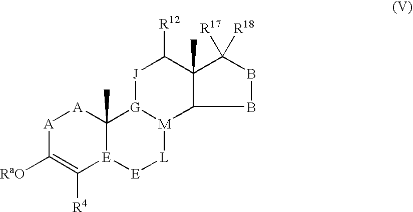

wherein

R⁴ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;

R¹⁷ and R¹⁸ are independently selected from the group consisting of hydrogen, alkyl, hydroxy, alkenyl and alkynyl; or R¹⁷ and R¹⁸ together form a ketal or keto group; or R¹⁷ and R¹⁸, together with the C₁₇ carbon to which they are attached, form the α-oriented or β-oriented cyclic structure:

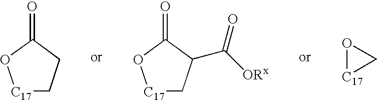

where R^{x} is alkyl; and

the substituents R^{a}, R¹², A-A, B-B, G-J, D-D, E-E and L-M are as defined above in Formula I.

The oxidation produces a product mixture comprising a steroid product corresponding to a compound of Formula VI:

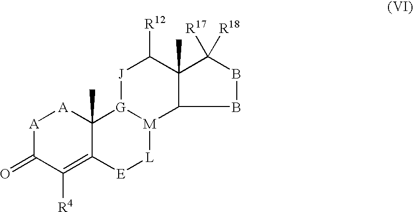

wherein

R¹⁷, R¹⁸ and R^{x} are as defined above in Formula V; E-L represents the group —CHR⁶—CHR⁷— or —CR═CR⁷—, where R⁶ and R⁷ are independent, R⁶ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy, and R⁷ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, heteroaryl, heterocyclyl, aryloxy, furyl and substituted furyl;

M-G represents the group:

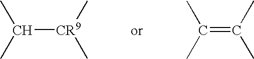

where R⁹ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and

the substituents R⁴, R¹², A-A, B-B and G-J are as defined in Formula I.

The oxidation reaction is summarized as shown in Reaction Scheme D:

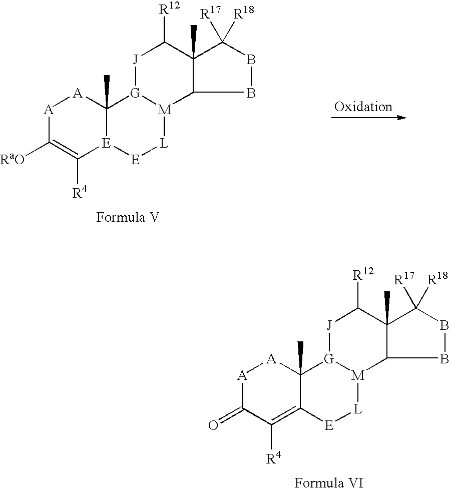

In a preferred embodiment, the oxidation process comprises oxidizing an enol ether substrate corresponding to the compound of Formula V-A to prepare a product mixture comprising a dienone steroid compound corresponding to a compound of Formula VI-A. As used herein the compound of Formula VI-A corresponds to the compound of Formula VI wherein the substituents R¹⁷ and R¹⁸ together with the C₁₇ carbon to which they are attached form a spirolactone group, as shown in Reaction Scheme D1:

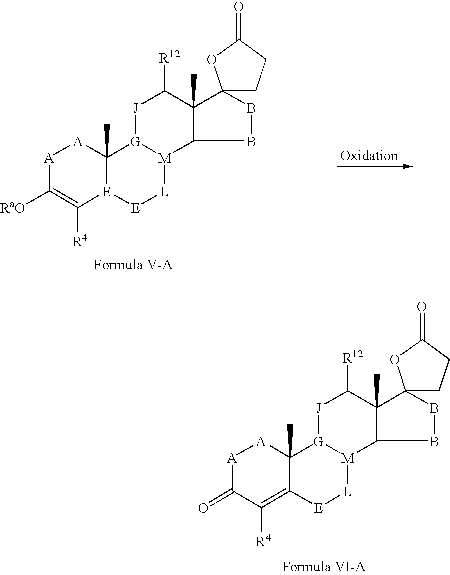

Preferably, the conditions and reactants of the oxidation reaction as described herein are selected such that the process shown above in Reaction Scheme D1 produces a product mixture comprising the β-oriented dienone steroid compound

corresponding to Formula VI-B:

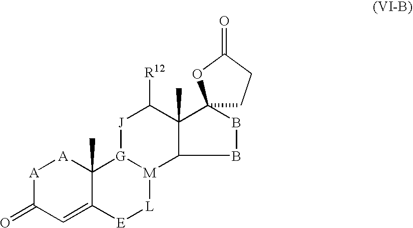

In a preferred embodiment, the steroid substrate of the oxidation reaction comprises a steroid compound selected from the group consisting of:

A process wherein the steroid substrate is selected from the group consisting of

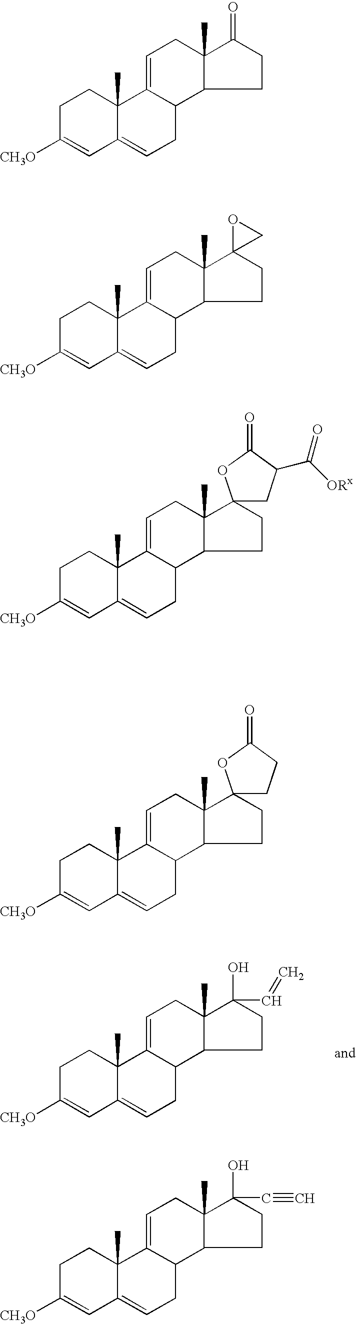

wherein R^{x} is alkyl.

In a further preferred embodiment, the steroid substrate of the oxidation reaction is a compound selected from the group consisting of:

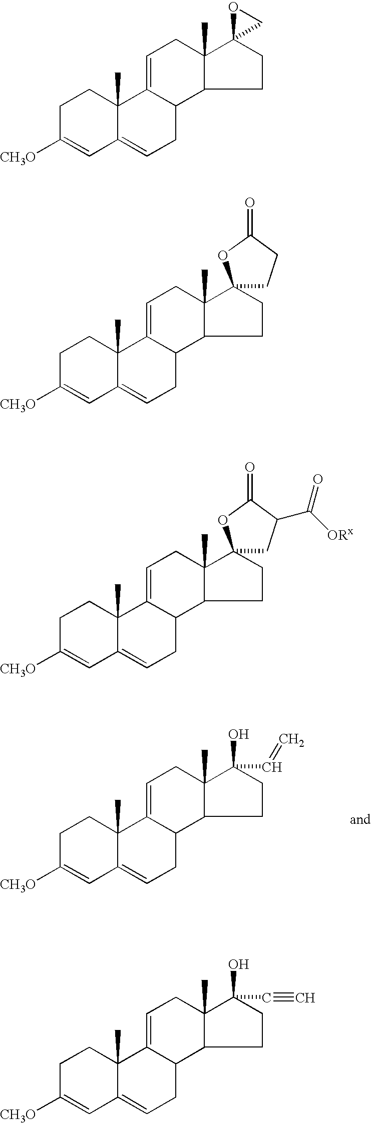

wherein R^{x} is alkyl.

Preferably, the oxidation reaction produces a product mixture comprising a steroid compound selected from the group consisting of:

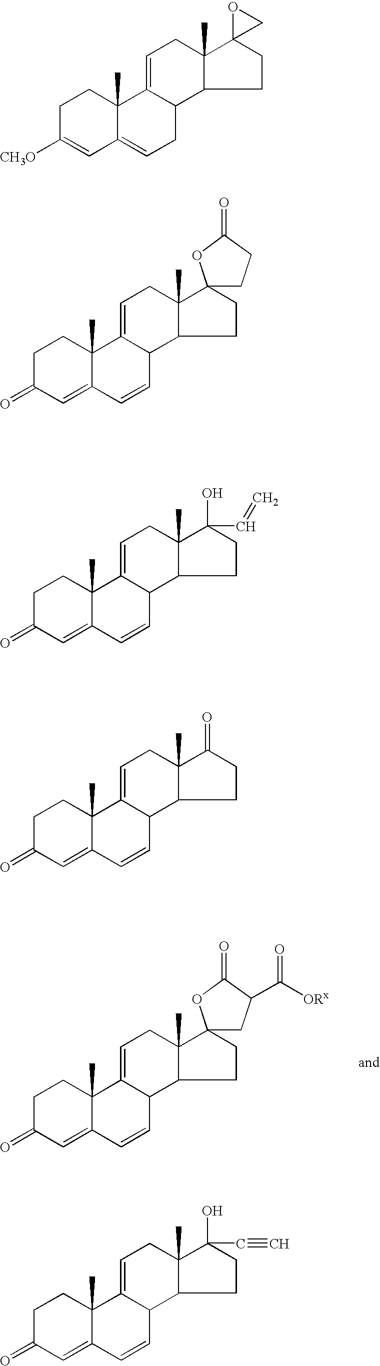

wherein R^{x} is alkyl.

Even more preferably, the conditions and reactants of the oxidation reaction are selected such that the reaction is effective in producing a steroid product comprising a compound selected from the group consisting of:

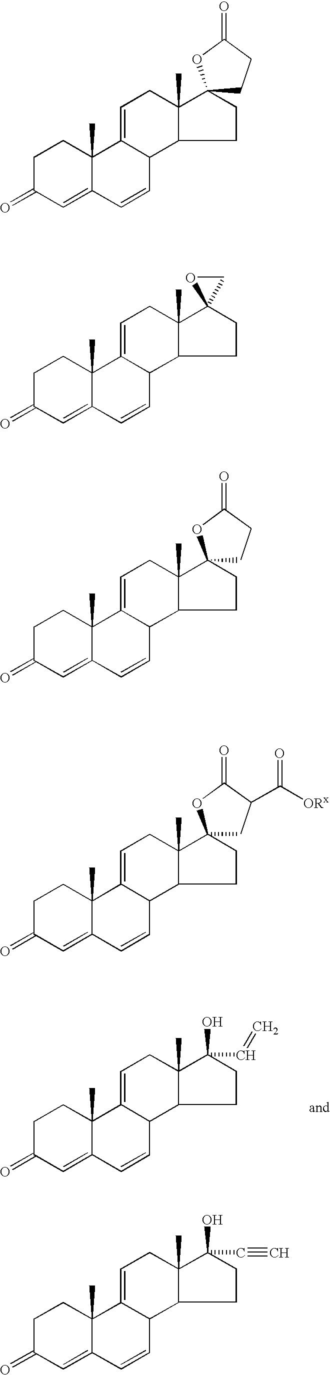

In general, Applicants have found that the oxidation may be completed using either of two methods:

1. Halogenation/Dehydrohalogenation

The first method for oxidizing the enol ether substrate of Formula V comprises contacting the enol ether substrate with a source of a halogen and water to produce a halogenated intermediate at the C₆ position. Preferably, the source of the halogen is electrophilic, such as an electrophilic source of chlorine or bromine. The halogenated intermediate is then dehydrohalogenated by contacting the intermediate with a base, thereby forming the steroid product of Formula VI.

2. Hydride Transfer

The second method for oxidizing the enol ether substrate comprises contacting an enol ether substrate corresponding to Formula V with an oxidizing agent in the presence of water to produce a steroid compound corresponding to Formula VI. Preferably, the enol ether substrate is contacted with an amount of oxidizing agent which is in excess of the stoichiometric amount of oxidizing agent required for the oxidation of the enol ether substrate. In particular, it is preferred to contact the enol ether substrate with about 1.01 to about 1.50 molar equivalents of oxidizing agent, more preferably with about 1.01 to about 1.25 molar equivalanets of oxidizing agent, and most preferably with about 1.01 to about 1.05 molar equivalents of oxidizing agent.

Preferably, the enol ether substrate and the oxidizing agent are contacted in the presence of water and a solvent. Suitable solvents include dimethylformamide, acetonitrile, methanol, acetone, methylene chloride and mixtures thereof. Especially preferred solvents include methylene chloride and mixtures of methylene chloride and methanol. For example, in a preferred embodiment, the enol ether substrate and the oxidizing agent are contacted in the presence of water and a mixture of methylene chloride and methanol.

Suitable oxidizing agents for use in the oxidation reaction include, for example, dichlorodicyanobenzoquinone (DDQ), o-chloranil (3,4,5,6-tetrachloro-o-benzoquinone), p-chloranil (2,3,5,6-tetrachloro-p-benzoquinone) and mixtures thereof. Typically, prior art processes for the oxidation of steroid substrates have preferred DDQ as an oxidizing agent over o-chloranil or p-chloranil as DDQ is generally known to be a stronger oxidant which allows for less side reactions during the oxidation of the steroid substrate. For example, although chloranil oxidizing agents are substantially less expensive than DDQ, chloranil oxidizing agents may often produce oxidation by-products such as substituted dihydroquinone compounds which can be problematic and/or tedious to remove from the product mixture, especially in a commercial setting. However, as part of the present invention, Applicants have discovered more efficient methods for the removal of substituted dihydroquinone by-products and the isolation of the steroid product from the oxidation reaction product mixture as described below, thereby providing a more efficient and cost-effective process for oxidizing enol ether steroid substrates.

Although not necessary or critical to the invention, Applicants have discovered that a preferred order of addition may involve introducing the steroid substrate and the oxidizing agent to the reaction as solids before introducing the water and/or solvent. Without being held to a particular theory, charging the solid reactants before adding water or solvent has been found to result in increased reaction rates and/or less product impurities which would suggest that such an order of addition be preferred, especially for commercial applications of the process. Further, when the oxidation reaction includes the use of both water and solvent, it may alternatively be more preferred to pre-mix the water and solvent to prepare a water/solvent mixture prior to contacting the enol ether substrate and the oxidizing agent.

Thus, in certain embodiments, preferred orders of addition for the reactants may be described as follows. In a first embodiment, the oxidation process comprises introducing the steroid substrate and the oxidizing agent into a reaction zone and thereafter contacting the steroid substrate and the oxidizing agent with a solvent and water in the reaction zone to prepare a product mixture comprising the steroid compound of Formula VI. In another embodiment, the process comprises preparing a substrate pre-mixture comprising the steroid substrate and the oxidizing agent. The substrate pre-mixture is then contacted with the solvent and water to prepare a product mixture comprising the steroid compound of Formula VI. Finally, in a third embodiment, the oxidation process comprises contacting the steroid substrate and the oxidizing agent with a pre-mixed reaction medium comprising solvent and water.

Experience to date with enol ether oxidation reactions using chloranil as the oxidizing agent has shown unreacted chloranil to be a source of impurities in the product mixture. Thus, in some embodiments of the process of the present invention, it may be preferred to contact the product mixture with a reducing agent in order to quench the reaction, thereby removing unreacted chloranil. Suitable reducing agents for contacting the oxidation reaction product mixture include sulfite, metabisulfite and mixtures thereof, with metabisulfite being preferred. For example, in a particularly preferred embodiment, the product mixture is contacted with a fresh metabisulfite solution prior to recovering the steroid product from the product mixture.

Further, as described above, experience to date suggests that substituted dihydroquinone by-products are a primary impurity associated with enol ether oxidation reactions using chloranil as the oxidizing agent. For example, in a preferred embodiment, the process of the present invention comprises oxidizing the steroid substrate of Formula V by contact with p-chloranil in the presence of water and a solvent, preferably a solvent comprising a mixture of methylene chloride and methanol. The process produces a reaction mixture comprising the steroid product of Formula VI and, typically, a substituted dihyroquinone by-product. Therefore, in certain preferred embodiments of the present invention, it may be desirable to further remove the dihydroquinone by-products from the product mixture prior to recovering the steroid product.

Thus, in a preferred embodiment, the process of the invention further includes removing dihydroquinone by-products from the product mixture by precipitation, preferably by contacting the product mixture with water. In a preferred embodiment wherein methylene chloride and methanol are used as the solvent system of the oxidation reaction, contacting the product mixture with water typically produces a two-phase system wherein methanol is partitioned substantially to the aqueous phase and the dihydroquinone by-products precipitate in the organic phase, thereby allowing the dihydroquinone by-products to be removed from the product mixture by filtering the organic phase or, alternatively, filtering the entire bi-phase.

Experience to date has further suggested that the product mixture may still contain residual dihydroquinone by-products even after precipitating dihydroquinone by-products from the product mixture as described above. Therefore, additional preferred embodiments of the present invention may further involve removing residual dihydroquinone by-products from the product mixture. Preferably, the residual dihydroquinone by-products can be removed from the product mixture by precipitation, for example, by contacting the product mixture with a base, and preferably by contacting the product mixture with a base under anhydrous conditions or essentially in the absence of a significant aqueous phase. For example, without being held to a particularly theory, experience to date suggests that contacting the product mixture with a base under essentially anhydrous conditions or in the absence of a significant aqueous phase is effective in efficiently precipitating dihydroquinone by-product salts in the product mixture. In contrast to previous experience with precipitating dihydroquinone by-products from the product mixture with water or aqueous acids or bases wherein precipitation of the dihydroquinone by-products with aqueous base produced gelatinous solids which were tedious or ineffective for practice on a commercial scale, Applicants have surprisingly discovered that contacting the product mixture with a base under essentially anhydrous conditions or in the absence of a significant aqueous phase causes efficient precipitation of dihydroquinone by-product satls which may be more easily removed from the product, for example by rapid filtration. Further, in accordance with the present invention, Applicants have also discovered that contacting the product mixture with a base under anhydrous conditions has an additional advantage in that it provides a general method for precipitating dihydroquinone by-products even when the desired product compound contains functional groups that are typically susceptible to reaction by bases under other conditions. Therefore, in certain preferred embodiments, the present invention provides a more effective process for the convenient and efficient removal of dihydroquinone by-products.

Suitable bases for use in removing dihydroquinone by-products from the product mixture include alkali metal hydroxides such as potassium hydroxide, sodium hydroxide, lithium hydroxide and mixtures thereof. Preferably, the base comprises potassium hydroxide, more preferably solid particulate potassium hydroxide. For example, as described above, experience to date suggests that potassium hydroxide, particularly solid particulate potassium hydroxide, reacts with dihydroquinone in the product mixture under heterogeneous conditions and in the absence of a significant aqueous phase to form insoluble dihydroquinone salts, which are thereby easily removed by filtration. After final removal of the dihydroquinone salt, the steroid product may be recovered from the product mixture by precipitation.

In a preferred embodiment, after removal of the dihydroquinone by-product as described above and before recovery of the steroid product from the product mixture, the process further comprises washing the product mixture with water to remove any residual base or dihydroquinone. Preferably, the steroid product is then recovered from the product mixture, most preferably by precipitation. It is important to note that the steroid product is very soluble in methylene chloride. Thus, when the product mixture comprises methylene chloride as a solvent, it is often preferred to replace the methylene chloride solvent with a suitable solvent such as methanol or water for precipitation of the steroid product. For example, in a preferred embodiment, methanol is added to the product mixture and methylene chloride is removed from the product mixture by distillation. After removal of the methylene chloride, the steroid product is recovered by precipitation, preferably by contacting the product mixture with water. In certain preferred embodiments, the recovered steroid product may be further washed with water, methanol or mixtures thereof, preferably a mixture of water and methanol. Finally, the process of the invention may still further comprise drying the recovered steroid product by any means generally known in the art.

Overall Process

In a particularly preferred embodiment, the above steps are combined in a process for the preparation of a dienone steroid compound of Formula VI-A. The overall reaction is summarized in Reaction Scheme E below.

In a particularly preferred embodiment, the present invention is directed to a process for the preparation of (17α)-17-hydroxy-3-oxo-Pregna-4,6,9(11)-triene-21-carboxylic acid γ-lactone (i.e., Δ⁹⁽¹¹⁾-canrenone). The process is shown below in Reaction Scheme F.

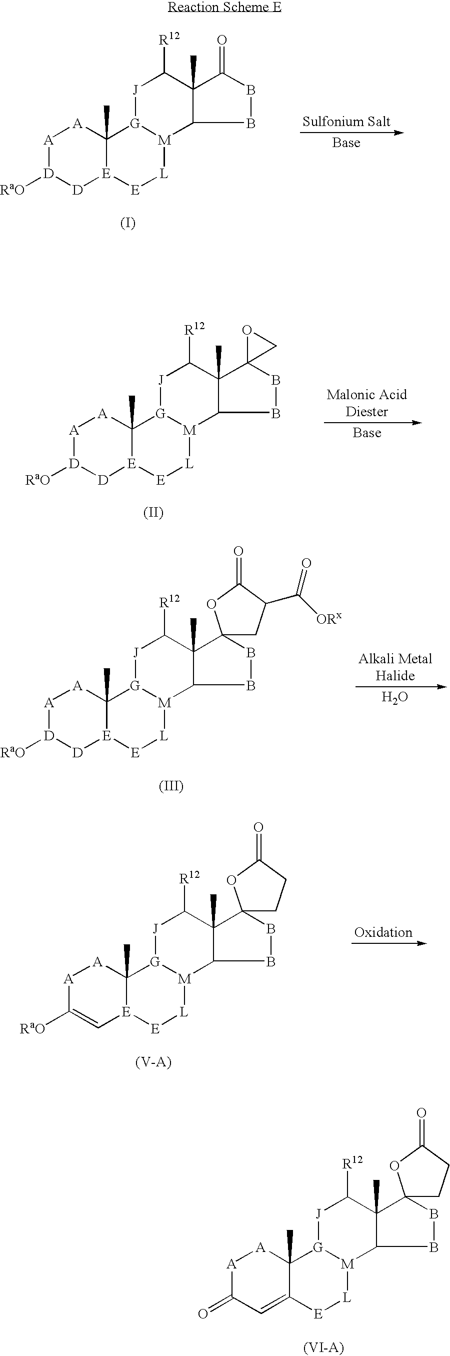

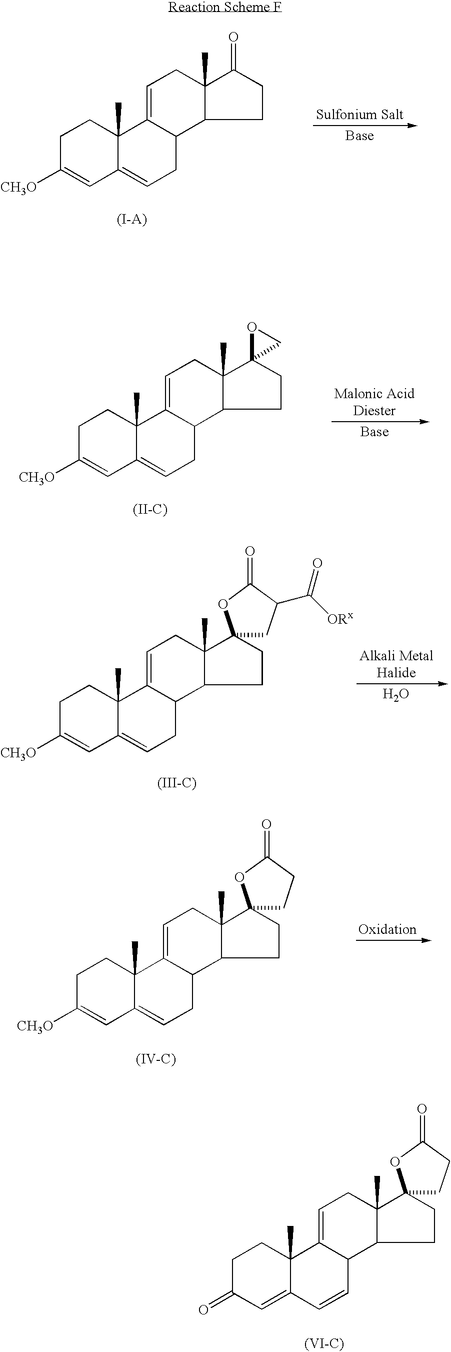

The process generally comprises contacting a steroid substrate comprising 3-methoxy-androsta-3,5,9(11)-trien-17-one with a base and a solvent medium containing a sulfonium salt to produce an oxirane intermediate compound, (17β)-3-methoxy-spiro[androsta-3,5,9(11)-17,2′-oxirane]. The oxirane intermediate compound is subsequently contacted with a malonic acid diester and a base in the presence of a solvent to form a dicarboxylate steroid intermediate, (17α)-17-hydroxy-3-methoxy-pregna-3,5,9(11)-triene-21,21-dicarboxylic acid ethyl ester γ-lactone. The dicarboxylate steroid intermediate is then decarboxylated by contact with an alkalki metal halide and water to form an enol ether steroid compound, (17α)-pregna-3,5,9(11)-triene-21-carboxylic acid γ-lactone. The enol ether steroid compound is then oxidized, preferably by contact with an oxidizing agent in the presence of water, to produce the Δ^{9,11} canrenone product.

Further, as described above and shown in Reaction Scheme G, another embodiment of the present invention is directed to a novel process for the preparation of methyl hydrogen 9,11α-epoxy-17α-hydroxy-3-oxopregn-4-ene-7α,21-dicarboxylate, γ-lactone (i.e., eplerenone or epoxymexrenone).

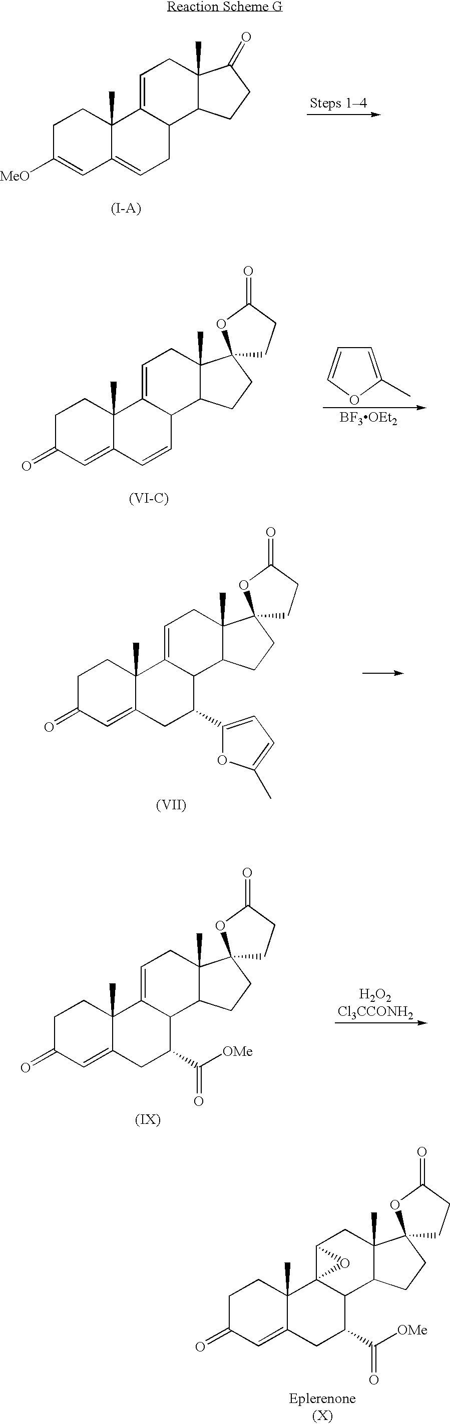

The process comprises preparing (17α)-17-hydroxy-3-oxo-Pregna-4,6,9(11)-triene-21-carboxylic acid γ-lactone (i.e., Δ⁹⁽¹¹⁾-canrenone) as described above in Reaction Scheme F (shown as Steps 1-4 in Reaction Scheme G). The Δ⁹⁽¹¹⁾-canrenone is then contacted with an alkyl furan and a Lewis acid to produce a 7α-furyl intermediate compound of Formula VII. The 7α-furyl intermediate compound of Formula VII is converted to a 7α-methoxycarbonyl intermediate compound of Formula IX, preferably by oxidizing the 7α-furyl intermediate compound of Formula VII, which is then converted to the epoxymexrenone steroid product. Processes for the preparation of eplerenone generally, and processes for the conversion of (17α)-17-hydroxy-3-oxo-Pregna-4,6,9(11)-triene-21-carboxylic acid γ-lactone (i.e., Δ⁹⁽¹¹⁾-canrenone) to eplerenone in particular, are more fully described in U.S. patent application Ser. No. ______, entitled “Processes To Prepare Eplerenone,” which was filed on even date herewith and the text of which is hereby incorporated herein by reference in its entirety.

### EXAMPLES

The following examples are simply intended to further illustrate and explain the present invention. This invention, therefore, should not be limited to any of the details in these examples.

As used in the examples, the steroid substrate abbreviated as “2DM” refers to the steroid compound 3-methoxy-androsta-3,5,9(11)-trien-17-one.

### Example 1

### Preparation of (17β)-3-methoxy-spiro[androsta-3,5,9(11)-17,2′-oxirane]

A solution of trimethylsulfonium methylsulfate in DMSO (TMSMS/DMSO solution) was prepared by charging DMSO (300 mL) and dimethyl sulfate (69.7 g) to a 500 mL round bottom flask equipped with a magnetic stirrer and an addition funnel. Dimethyl sulfide (37.5 g) was then charged to the reactor over a period of 15 minutes with agitation. The mixture was stirred for another 4 hours at a 50° C. A 1 L reactor was charged with 2DM (100 g), pulverized KOH (32.4 g) and THF (400 mL) to prepare a slurry, which was maintained at a temperature of between 5° and 20° C. with adequate agitation. The TMSMS/DMSO solution prepared above was then charged to the slurry and the mixture was heated to 65° C. After two hours of heating, the mixture was sampled for reaction completion. The reaction was deemed complete when the area percent of 2DM was 0.5% or less. After the reaction was complete, the reaction mixture was distilled under vacuum (45 mm Hg at 40° C.) to remove by-product dimethyl sulfide. Distillation was continued until 300 mL of solvent was removed. After distillation, dilution water (500 mL) was charged to the DMSO/product slurry over 30 minutes at 40° C. The DMSO/product slurry and water mixture was held for 30 minutes and filtered. The filtered product was then re-slurried in water (500 mL) at 40° C. The water re-slurry wash was repeated two additional times. The wet product was then dried under vacuum at 60° C. to afford 102.6 g of a product comprising 94.5% by weight of (17β)-3-methoxy-spiro[androsta-3,5,9(11)-17,2′-oxirane].

### Example 2

### Preparation of (17β)-3-methoxy-spiro[androsta-3,5,9(11)-17,2′-oxirane]

A 1 L reactor was charged with 2DM (25.0 g), THF (120 g), 22% trimethylsulfonium methylsulfate in DMSO (TMSMS in DMSO, 132 g) at 3° to 5° C. under an atmosphere of nitrogen. A 1 M KOt-Bu/THF solution (138 g) was added within a one hour period at 3° to 15° C. and stirred at 3° to 15° C. for 1 hour. The reaction was completed (<0.5% area 2DM) in one hour. Water (10 g) was added and the resulting mixture was distilled under vacuum (45 mm Hg at 40° C.) to remove by-product dimethyl sulfide. Distillation was continued until 200 g of solvent was removed. After distillation, 200 g of dilution water was charged to the DMSO/product slurry over 30 minutes at 40° C. The DMSO/product slurry and water mixture was held for 30 minutes and filtered. The filtered product was then re-slurried twice in water (125 ml) at 40° C. The cake was washed with 3-5 volumes of MeOH at room temperature for further purification. The wet product was then dried under vacuum at 60° C. to produce 88% assay adjusted wt yield with product >99% pure.

### Example 3

### Preparation of (17β)-3-methoxy-spiro[androsta-3,5,9(11)-17,2′-oxirane]

A solution of trimethylsulfonium methylsulfate in DMSO (TMSMS/DMSO solution) was prepared by charging DMSO (300 mL) and diethylsulfide (37.5 g) to a 500 mL round bottom flask equipped with a magnetic stirrer and an addition funnel. Dimethyl sulfate (69.7 g) was then charged to the reactor over a period of 15 minutes with agitation. The mixture was stirred for another 4 hours at 50° C. A 1 L reactor was charged with 2DM (100 g), pulverized KOH (32.4 g) and THF (400 mL) to prepare a slurry, which was maintained at a temperature of from 5° to 20° C. with adequate agitation. The TMSMS/DMSO solution prepared above was then charged to the slurry and the mixture was heated to 65° C. After two hours of heating, the mixture was sampled for reaction completion. The reaction was deemed complete when the area percent of 2DM was 0.5% or less. After the reaction was complete, the reaction mixture was distilled under vacuum (45 mm Hg at 40° C.) to remove by-product dimethyl sulfide. Distillation was continued until 300 mL of solvent was removed. After distillation, dilution water (500 mL) was charged to the DMSO/product slurry over 30 minutes at 40° C. The DMSO/product slurry and water mixture was held for 30 minutes and filtered. The filtered product was then re-slurried in water (500 mL) at 40° C. The water re-slurry wash was repeated two additional times. The wet product was then dried under vacuum at 60° C. overnight to isolate 102.6 g of a product comprising 94.5% by weight oxirane intermediate.

### Example 4

### Preparation of (17β)-3-methoxy-spiro[androsta-3,5,9(11)-17,2′-oxirane]

A solution of trimethylsulfonium methylsulfate in DMSO (TMSMS/DMSO solution) was prepared by charging DMSO (300 mL) and diethylsulfide (37.5 g) to a 500 mL round bottom flask equipped with a magnetic stirrer and an addition funnel. Dimethyl sulfate (69.7 g) was then charged to the reactor over a period of 15 minutes with agitation. The mixture was stirred for another 4 hours at 50° C. A 1 L reactor was charged with 2DM (100 g), pulverized KOH (32.4 g) and THF (400 mL) to prepare a slurry, which was maintained at a temperature of from 5° to 20° C. with adequate agitation. The TMSMS/DMSO solution prepared above was then charged to the slurry and the mixture was heated to 65° C. After two hours of heating, the mixture was sampled for reaction completion. The reaction was deemed complete when the area percent of 2DM was 0.5% or less. After the reaction was complete, the reaction mixture was distilled under vacuum (45 mm Hg at 40° C.) to remove by-product dimethyl sulfide. Distillation was continued until 300 mL of solvent was removed. After distillation, dilution water (500 mL) was charged to the DMSO/product slurry over 30 minutes at 40° C. The DMSO/product slurry and water mixture was held for 30 minutes and filtered. The filtered product was then re-slurried twice in water (500 mL) at 40° C. Finally the product was washed with methanol (300 mL) by re-slurry for 2 hours at 20° C. The wet product was then dried under vacuum at 60° C. overnight to isolate 93.5 g of a product comprising 98.5% by weight oxirane intermediate.

### Example 5

### Preparation of (17β)-3-methoxy-spiro[androsta-3,5,9(11)-17,2′-oxirane]

A 1 L reactor was charged with 2DM (100 g), pulverized KOH (32.4 g), TMSMS (104 g), THF (400 mL) and DMSO (300 mL). The mixture was heated to 65° C. with adequate agitation. After two hours of heating, the mixture was sampled for reaction completion. The reaction was deemed complete when the area percent of 2DM was 0.5% or less. After the reaction was complete, the reaction mixture was distilled under vacuum (45 mm Hg at 40° C.) to remove by-product dimethyl sulfide. Distillation was continued until 300 mL of solvent was removed. After distillation, dilution water (500 mL) was charged to the DMSO/product slurry over 30 minutes at 40° C. The DMSO/product slurry and water mixture was held for 30 minutes and filtered. The filtered product was then re-slurried twice in water (500 mL) at 40° C. Finally the product was washed with methanol (300 mL) by re-slurry for 2 hours at 20° C. The wet product was then dried under vacuum at 60° C. overnight to isolate 93.5 g of a product comprising 98.5% by weight oxirane intermediate.

### Example 6

### Preparation of (17α)-17-hydroxy-3-methoxy-pregna-3,5,9(11)-triene-21,21-dicarboxylic Acid Ethyl Ester γ-lactone

A 1 L reactor was purged with nitrogen and oxirane intermediate (50 g) was charged to the reactor followed by anhydrous ethanol (138 g), diethyl malonate (46 g) and a solution of 21% sodium ethoxide (88 g). The mixture was heated to reflux (approximately 79° C. to 81° C.) for four hours and sampled for reaction completion. The reaction was deemed complete when the reaction mixture contained less than 1.0% oxirane intermediate (as determined by normalized area percent). After reaction completion, the reaction mixture was cooled to a temperature of from 40° to 70° C. and the reaction mixture was neutralized by charging glacial acetic acid (15.5 g) to the reaction mixture over a period of 30 minutes followed by water (25 g). The solution was cooled to 15° C. and then additional water (225 g) was charged in 60-90 minutes. The slurry was held at 15° C. for another 30 minutes and the product was then isolated by filtration and washed by re-slurry with 30% MeOH/water V/V (500 mL) to afford a wet product cake. The product was dried under vacuum at 60° C. overnight. The reaction afforded 65.5 g of product representing a 96% by weight yield (unadjusted for assay).

### Example 7

### Preparation of (17α)-17-hydroxy-3-methoxy-pregna-3,5,9(11)-triene-21,21-dicarboxylic Acid Ethyl Ester γ-lactone

A 1 L reactor was purged with nitrogen and oxirane intermediate (50 g) was charged to the reactor followed by DMF (88 g), diethyl malonate (46 g) and a solution of 21% sodium ethoxide (88 g). The mixture was heated to 800 to 95° C. for 6 hours and sampled for reaction completion. The reaction was deemed complete when the reaction mixture contained less than 1.0% oxirane intermediate (as determined by normalized area percent). After reaction completion, the temperature was reduced to 40° C. The reaction mixture was neutralized with glacial acetic acid (15.5 g) in 25 minutes, followed by water (920 mL) in 30 minutes. The product was then isolated by filtration and washed by re-slurry in water (400 mL). The cake was dried under vacuum at 60° C. overnight to give 61.6 g of product representing a 90% by weight yield (unadjusted for assay).

### Example 8

### Preparation of (17α)-pregna-3,5,9(11)-triene-21-carboxylic Acid γ-lactone

A 1 L reactor was purged with nitrogen and then dicarboxylate intermediate (64.0 g), sodium chloride (13.29 g), dimethylformamide (192.0 mL) and water (4.1 mL) were charged to the reactor. The mixture was heated to reflux (135° to 142° C.) and held for 8 hours before sampling for reaction completion. The reaction was deemed complete when the amount of dicarboxylate intermediate remaining in the reaction mixture was 0.5% or less (as calculated by normalized area %). After completion, the reaction temperature was reduced to 40° C. and dilution water (256 mL) was charged over a period of 30 minutes. The product slurry was then cooled to 20° C. and held for another 30 minutes before isolation. The product was isolated by filtration and then washed by re-slurry with water (256 mL) followed by a displacement wash of 154 g of methanol. The product was dried under vacuum at 60° C. overnight to afford 53.0 g of an off-white solid.

### Example 9

### Preparation of (17α)-17-hydroxy-3-oxo-Pregna-4,6,9(11)-triene-21-carboxylic Acid γ-lactone (i.e., Δ⁹⁽¹¹⁾-canrenone)

Enol ether substrate (100.0 g) and chloranil (72.2 g) were charged to a 1 L reactor followed by a pre-mixed solution of methylene chloride (200 mL), methanol (120 mL) and water (40 mL) while stirring. The suspension was heated to reflux (42° C.) for 2 hours over which time the mixture changed from a yellow suspension to an red-brown homogeneous solution. The reaction was checked for completion using LC. After the reaction was complete, the solution was cooled to room temperature and a solution of 20% sodium metabisulfate (30 mL) was added. The resulting mixture was stirred for 30 minutes. Water (490 mL) was added and the resulting biphase was stirred for 30 minutes. The dihydroquinone byproduct precipitated in the organic phase. The entire biphase was filtered to separate the precipitated dihydroquinone byproduct and the cake was washed twice with methylene chloride (70 mL each wash). The residual aqueous phase was removed from the filtrate and the organic phase was transferred back to the reactor for removal of the remaining dihydroquinone byproduct. The remaining byproduct was removed by contacting the residual organic phase with pulverized KOH (6.6 g) suspended in methylene chloride (70 mL) with stirring. The suspension was stirred for 1 hour and filtered to remove the dihydroquinone salt byproducts. The byproduct cake was washed twice with methylene chloride (66 mL each wash). Steroid product present in the filtrate was then isolated by crystallization. Prior to crystallization, the organic phase from above was washed twice with water (300 mL each wash). The mixture was then distilled at atmospheric pressure to remove methylene chloride. Methanol (379 mL) was then added and distillation was continued until the pot temperature reached 650 to 75° C. Additional methanol (35 mL) was added and the mixture was cooled to 40° C. Water (500 mL) was added over 1 hour. The suspension was then cooled within the range of 3° C. to 15° C. and held for 30 minutes. The solids were filtered and washed with a solution of methanol/water (1:1 v/v, 250 mL). Solids were dried at 70° C. in a vacuum oven with a nitrogen bleed until constant weight was obtained. Isolated 88.0 g product (92.1% molar yield unadjusted for assay).

### Example 10

### Preparation of (17β)-17-hydroxy-3-oxo-Pregna-4,6,9(11)-triene-21-carboxylic Acid γ-lactone (i.e., 17-epi-Δ⁹⁽¹¹⁾-canrenone)

Enol ether substrate (61.3 g) and chloranil (44.2 g) were charged to a 1 L reactor followed by a pre-mixed solution of methylene chloride (123 mL), methanol (74 mL) and water (25 mL) while stirring. The suspension was heated to reflux (42° C.) for 1 hour over which time the mixture changed from a yellow suspension to an red-brown homogeneous solution. The reaction was checked for completion using LC. After the reaction was complete, the solution was cooled to room temperature and a solution of 20% sodium metabisulfate (19 mL) was added and the resulting mixture was stirred for 10 minutes. Water (300 mL) was added and the resulting biphase was stirred for 30 minutes. The dihydroquinone byproduct precipitated in the organic phase. The entire biphase was filtered to separate the precipitated dihydroquinone byproduct and the cake was washed twice with methylene chloride (37 mL each wash). The residual aqueous phase was removed from the filtrate and the organic phase was transferred back to the reactor for removal of the remaining dihydroquinone byproduct. The remaining byproduct was removed by contacting the residual organic phase with pulverized KOH (4.2 g) suspended in methylene chloride (45 mL) with stirring. The suspension was stirred for 1 hour and filtered to remove the dihydroquinone salt byproducts. The byproduct cake was washed twice with methylene chloride (37 mL each wash). Steroid product present in the filtrate was then isolated by crystallization. Prior to crystallization, the organic phase from above was washed twice with water (185 mL each wash). The mixture was then distilled at atmospheric pressure to remove methylene chloride. Methanol (232 mL) was then added and distillation was continued until the pot temperature reached 65° to 75° C. Additional methanol (74 mL) was added and the mixture was cooled to 58° C. Water (307 mL) was added over 1 h while cooling to room temperature. The suspension was then cooled within the range of 3° to 15° C. and held for 30 minutes. The solids were filtered and washed with a solution of methanol/water (1:1 v/v, 150 mL). Solids were dried at 70° C. in a vacuum oven with a nitrogen bleed until constant weight was obtained. Isolated 51.2 g product (87.5% molar yield unadjusted for assay).

### Example 11

### Preparation of Androsta-4,6,9(11)-triene-3,17-dione

Enol ether substrate (4.2 g) and chloranil (3.7 g) were charged to a 100 mL reactor followed by a pre-mixed solution of acetone (45 mL), and water (2.5 mL) while stirring. The suspension was stirred at room temperature for 1 hour over which time the mixture changed from a yellow suspension to an red-brown homogeneous solution. The reaction was checked for completion using LC. After the reaction was complete, the solution was cooled to room temperature and a solution of 20% sodium metabisulfate (5 mL) was added and the resulting mixture was stirred for 10 minutes. Acetone was removed under reduced pressure and methylene chloride (25 mL) was added. The biphase was filtered to separate the precipitated dihydroquinone byproduct and the cake was washed twice with methylene chloride (5 mL each wash). The residual aqueous phase was removed from the filtrate and the organic phase was transferred back to the reactor for removal of the remaining dihydroquinone byproduct. The remaining byproduct was removed by contacting the residual organic phase with pulverized KOH (0.5 g) suspended in methylene chloride (10 mL) with stirring. The suspension was stirred for 30 minutes and filtered. The filtered solid was washed twice with methylene chloride (5 mL each wash). Steroid product present in the filtrate was then isolated by crystallization. Prior to crystallization, the organic phase from above was washed twice with water (15 mL each wash). The mixture was then distilled at atmospheric pressure to remove methylene chloride. Methanol (20 mL) was then added and distillation was continued until the pot temperature reached 65° to 75° C. Additional methanol (1.5 mL) was added and the mixture was cooled to 40° C. Water (21 mL) was added over 30 minutes while cooling to room temperature. The suspension was then cooled within the range of 3° to 15° C. and held for 30 minutes. The solids were filtered and washed with water (10 mL). Solids were dried under air in the filter until constant weight was obtained. Isolated 3.3 g product (82.7% molar yield unadjusted for assay).

### Example 12

### Preparation of (17β)-3-methoxy-spiro [androsta-4,6,9(11)-17,2′-oxirane]

Enol ether substrate (50.6 g) and chloranil (40.0 g) were charged to a 100 mL reactor followed by a pre-mixed solution of methylene chloride (100 mL), methanol (60 mL) and water (20 mL) while stirring. The suspension was heated to 35° C. for 1.5 hours over which time the mixture changed from a yellow suspension to an red-brown homogeneous solution. The reaction was checked for completion using LC. After the reaction was complete, the solution was cooled to room temperature and a solution of 20% sodium metabisulfate (15 mL) was added and the resulting mixture was stirred for 10 minutes. Water (250 mL) was added and the resulting biphase was stirred for 30 minutes. The dihydroquinone byproduct precipitated in the organic phase. The entire biphase was filtered to separate the precipitated dihydroquinone byproduct and the cake was washed twice with methylene chloride (30 mL each wash). The residual aqueous phase was removed from the filtrate and the organic phase was transferred back to the reactor for removal of the remaining dihydroquinone byproduct. The remaining byproduct was removed by contacting the residual organic phase with pulverized KOH (3 g) suspended in methylene chloride (10 mL) with stirring. The suspension was stirred for 20 minutes and filtered to remove the dihydroquinone salt byproducts. The byproduct cake was washed twice with methylene chloride (30 mL each wash). Steroid product present in the filtrate was then isolated by crystallization. Methylene chloride was removed under reduced pressure. Methanol (100 mL) was then added and then removed under reduced pressure. The product was isolated as a crystalline material and was dried under air in the filter until constant weight was obtained. Isolated 43.9 g product (92.6% molar yield unadjusted for assay).

### Example 13

### Preparation of (17α)-17-hydroxy-3-oxo-Pregna-4,6,9(11)-triene-21,21-dicarboxylic Acid Ethyl ester γ-lactone

Enol ether substrate (20.0 g) and chloranil (18.0 g) were charged to a 1 L reactor followed by a pre-mixed solution of methylene chloride (70 mL), methanol (23 mL) and water (10 mL) while stirring. The suspension was heated to reflux (42° C.) for 2 hours over which time the mixture changed from a yellow suspension to an red-brown homogeneous solution. The reaction was checked for completion using LC. After the reaction was complete, the solution was cooled to room temperature and an aqueous solution of 20% Na₂S₂O₅ (10 g) was added and the mixture was stirred for 30 minutes. Water (160 mL) was added and the resulting biphase was stirred for 30 minutes. The dihydroquinone byproduct precipitated in the organic phase. The entire biphase was filtered to separate the precipitated dihydroquinone byproduct and the cake was washed twice with methylene chloride (50 mL each wash). The residual aqueous phase was removed from the filtrate and the organic phase was transferred back to the reactor for removal of the remaining dihydroquinone byproduct. The remaining byproduct was removed by contacting the residual organic phase with pulverized KOH (3.6 g) with stirring. The suspension was stirred for 30 minutes and filtered to remove the dihydroquinone salt byproducts. The byproduct cake was washed twice with methylene chloride (50 mL each wash) and the resulting filtrate was washed twice with water (50 mL each wash). The organic phase was concentrated to afford the product as an off-white solid.

### Example 14

### Preparation of (17α)-17-hydroxy-3-oxo-Pregna-4,6,9(11)-triene-21-carboxylic Acid γ-lactone (i.e., Δ⁹⁽¹¹⁾-canrenone)

Enol ether substrate (50.1 g), acetone (200 mL) and water (50 mL) were charged to a 1-liter, 3-necked round-bottomed flask equipped with magnetic stirring. The resulting mixture was cooled to −4° C. and 1,3-dibromo-5,5-dimethylhydantoin (22.1 g) was added in a single charge while maintaining a temperature below 10° C. The reaction was checked for completion with LC. After completion, the reaction was quenched with ethyl vinyl ether (2.5 mL). The reaction was poured onto NaHCO₃ (100 mL of ½ sat. aq. solution) and ethyl acetate (150 mL) was added. The biphase was separated and the aqueous layer was extracted with ethyl acetate (100 mL). The organic phases were combined and washed twice with water (200 mL each wash). The solution was concentrated to approximately 100 g. DMF (25 mL) was added and the resulting solution was charged to a 500 mL, 3-necked round-bottomed flask containing DABCO (19.4 g) in DMF (50 mL) heated to 70° C. After the addition, residual material was rinsed into the reaction flask with additional DMF (75 mL). The reaction was heated to 70° C. for 2 hours then cooled to room temperature and poured onto water (200 mL). Methylene chloride (200 mL) was added and the biphase was separated. The aqueous phase was extracted with CH₂Cl₂ (100 mL). The combined organic layers were washed with 5% H₂SO₄ (200 mL) then water (200 mL). The organic layer was dried (MgSO₄), filtered and concentrated to afford an orange oil. Methanol (15 mL) was added to the oil and the mixture was heated to dissolve all solids and oils. The product crystallized and was isolated by filtration at 5° C. to afford 37.2 g of yellow solid (75% assay adjusted molar yield).

### Example 15

### Preparation of (17α)-17-hydroxy-3-oxo-Pregna-4,6,9(11)-triene-21-carboxylic Acid γ-lactone (i.e., Δ⁹⁽¹¹⁾-canrenone)

Enol ether substrate (5.0 g), acetone (20 mL) and water (5 mL) were charged to a 50 mL, 3-necked round-bottom flask equipped with a magnetic stirrer. The resulting mixture was cooled to −4° C. and 1,3-dibromo-5,5-dimethylhydantoin (2.2 g) was added in a single charge while maintaining the temperature below 10° C. The reaction was monitored by LC for completion. After completion, the reaction was quenched with ethyl vinyl ether (0.25 mL). The reaction was poured onto NaHCO₃ (10 mL of ½ sat. aq. solution) and ethyl acetate (15 mL) was added. The biphase was separated and the aqueous layer was extracted with ethyl acetate (10 mL). The organic phases were combined and washed twice with water (20 mL each wash). The solution was concentrated to approximately 10 g. DMF (2 mL) was added and the resulting solution was charged to a 50 mL, 3-necked round-bottomed flask containing Li₂CO₃/LiBr (1.3 g each) in DMF (5 mL) heated to 70° C. After the addition, residual material was rinsed into the reaction flask with additional DMF (8 mL). The reaction was heated to 70° C. for 2 hours then cooled to room temperature and poured onto water (25 mL). Methylene chloride (25 mL) was added and the biphase was separated. The aqueous phase was extracted with CH₂Cl₂ (10 mL). The combined organic layers were washed three times with water (25 mL each wash). The organic layer was dried (MgSO₄), filtered and concentrated to afford a yellow oil. Methanol (75 mL) was added to the oil and the mixture was heated to dissolve all solids and oils. The product crystallized and was isolated by filtration at 5° C. to afford 4.0 g of yellow solid (83% molar yield unadjusted for assay).

In view of the above, it will be seen that the several objects of the invention are achieved and other advantageous results attained. As various changes can be made in the above processes and compositions without departing from the scope of the invention, it is intended that all matter contained in the above description shall be interpreted as illustrative and not in a limiting sense.

What is claimed:

1. A process for the preparation of a steroid compound corresponding to the Formula II: 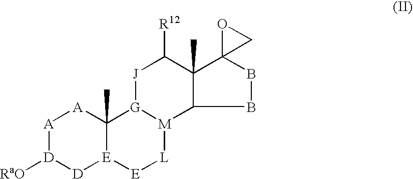
wherein:
R^{a} is alkyl;
R¹² is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
B-B represents the group —CHR¹⁵—CHR¹⁶— or an α-oriented or β-oriented cyclic group: 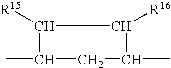
where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
G-J represents the group: 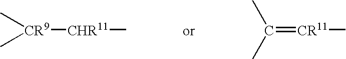
where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
D-D represents the group: 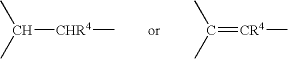
where R⁴ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
E-E represents the group: 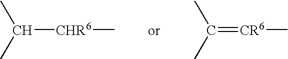
where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and
L-M represents the group: 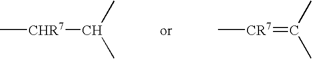
where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl,
the process comprising:
contacting a steroid substrate corresponding to the Formula I: 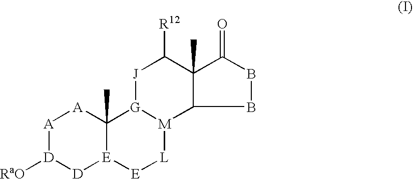
wherein the substituents R^{a}, R¹², A-A, B-B, D-D, E-E, G-J and L-M are as defined in Formula II, with a base and a solvent medium containing a sulfonium salt to produce a product mixture comprising the compound of Formula II.

2. A process as set forth in claim 1, wherein the product mixture comprises the β-oriented oxirane compound of Formula II in preference to the α-oriented oxirane compound of Formula II, said β-oriented oxirane compound of Formula II corresponding to the compound of Formula II-B: 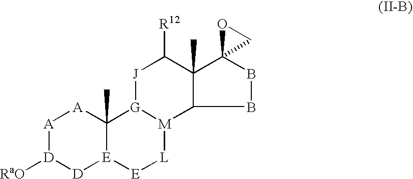
wherein the substituents R^{a}, R¹², A-A, B-B, D-D, E-E, G-J and L-M are as defined in Formula II.

3. A process as set forth in claim 2, wherein the solvent medium, the base and the reaction conditions are selected to yield said β-oriented oxirane compound in a ratio to the corresponding α-oriented oxirane compound of at least about 70:30.

4. A process as set forth in claim 2, wherein the solvent medium, the base and the reaction conditions are selected to yield said β-oriented oxirane compound in a ratio to the corresponding α-oriented oxirane compound of at least about 90:10.

5. A process as set forth in claim 2, wherein the solvent medium, the base and the reaction conditions are selected to yield said β-oriented oxirane compound in a ratio to the corresponding α-oriented oxirane compound of at least about 95:5.

6. A process as set forth in claim 1, wherein the process further comprises:
preparing a substrate pre-mixture comprising the steroid substrate and the base in a solvent medium; and
contacting the substrate pre-mixture with the solvent medium containing the sulfonium salt.

7. A process as set forth in claim 6 wherein the substrate pre-mixture is maintained at a temperature of less than about 15° C. before being contacted with the solvent medium containing the sulfonium salt.

8. A process as set forth in claim 6 wherein the substrate pre-mixture is maintained at a temperature of less than about 10° C. before being contacted with the solvent medium containing the sulfonium salt.

9. A process as set forth in claim 6 wherein the substrate pre-mixture is maintained at a temperature of less than about 5° C. before being contacted with the solvent medium containing the sulfonium salt.

10. A process as set forth in claim 6, wherein the solvent medium of the substrate pre-mixture comprises a solvent selected from the group consisting of dimethylsulfoxide, diethyl ether, dioxanes, diglyme, triglyme, dimethylformamide, tetrahydrofuran, dimethylacetamide, acetonitrile and mixtures thereof.

11. A process as set forth in claim 6, wherein the solvent medium containing the sulfonium salt comprises a solvent selected from the group consisting of dimethylsulfoxide, diethyl ether, dioxanes, diglyme, triglyme, dimethylformamide, tetrahydrofuran, dimethylacetamide, acetonitrile and mixtures thereof.

12. A process as set forth in claim 6, wherein the solvent medium containing the sulfonium salt and the solvent medium of the substrate pre-mixture are independently selected from the group consisting of dimethylsulfoxide, diethyl ether, dioxanes, diglyme, triglyme, dimethylformamide, tetrahydrofuran, dimethylacetamide, acetonitrile and mixtures thereof.

13. A process as set forth claim 12, wherein the sulfonium salt comprises a trimethylsulfonium salt.

14. A process as set forth in claim 13, wherein the sulfonium salt comprises trimethylsulfonium methyl sulfate.

15. A process as set forth in claim 13, wherein the solvent medium containing the sulfonium salt comprises dimethylsulfoxide.

16. A process as set forth in claim 15, wherein the solvent medium of the substrate pre-mixture comprises tetrahydrofuran.

17. A process as set forth in claim 6, wherein the process further comprises preparing the solvent medium containing the sulfonium salt.

18. A process as set forth in claim 17, wherein the solvent medium containing the sulfonium salt is prepared by contacting dimethyl sulfide with an alkanizing agent in the presence of the solvent medium.

19. A process as set forth in claim 18, wherein the alkanizing agent is selected from the group consisting of dimethyl sulfate or dimethyl iodide.

20. A process as set forth in claim 19, wherein the alkanizing agent comprises dimethyl sulfate.

21. A process as set forth in claim 18, wherein the solvent medium is selected from the group consisting of dimethylsulfoxide, diethyl ether, dioxanes, diglyme, triglyme, dimethylformamide, dimethylacetamide and mixtures thereof.

22. A process as set forth in claim 21, wherein the solvent medium comprises dimethylsulfoxide.

23. A process as set forth in claim 21, wherein the solvent medium containing the sulfonium salt comprises trimethylsulfonium methyl sulfate in dimethylsulfoxide.

24. A process as set forth in claim 21, wherein the solvent selected as the solvent medium containing the sulfonium salt and the solvent selected as the solvent medium of the substrate pre-mixture are independent, the solvent medium of the steroid substrate pre-mixture being selected from the group consisting of dimethylsulfoxide, diethyl ether, dioxanes, diglyme, triglyme, dimethylformamide, tetrahydrofuran, dimethylacetamide, acetonitrile and mixtures thereof.

25. A process as set forth in claim 24, wherein the solvent medium of the substrate pre-mixture comprises tetrahydrofuran.

26. A process as set forth in claim 6, wherein the base is selected from the group consisting of alkali metal hydroxides, alkali metal hydrides, t-butyl alkali metal alkoxides and alkaline earth metal hydroxides.

27. A process as set forth in claim 26, wherein the base is selected from the group consisting of KOH, NaOH, LiOH, KH, NaH, LiH and mixtures thereof.

28. A process as set forth in claim 27, wherein the base comprises a solid particulate.

29. A process as set forth in claim 28, wherein the base comprises potassium hydroxide.

30. A process as set forth in claim 6, wherein the molar ratio of base to sulfonium salt is from about 0.75:1 to about 1.5:1.

31. A process as set forth in claim 6, wherein the molar ratio of base to sulfonium salt is from about 0.9:1 to about 1.1:1.

32. A process as set forth in claim 6, wherein the process further comprises removing solvent from the product mixture by distillation.

33. A process as set forth in claim 6, wherein the process further comprises recovering a steroid product from the product mixture by precipitation, said recovered steroid product comprising the compound of Formula II.

34. A process as set forth in claim 33, wherein said precipitation comprises contacting the product mixture with water.

35. A process as set forth in claim 33, wherein the process further comprises washing the recovered steroid product.

36. A process as set forth in claim 35, wherein the recovered steroid product is washed by contacting said steroid product with water.

37. A process as set forth in claim 36, wherein the recovered steroid product is washed by contacting said steroid product with water at a temperature of at least about 25° C.

38. A process as set forth in claim 36, wherein the recovered steroid product is washed by contacting said steroid product with water at a temperature of at least about 40° C.

39. A process as set forth in claim 36, wherein the recovered steroid product is further washed by contacting said steroid product with an alcohol.

40. A process as set forth in claim 39, wherein the recovered steroid product is washed by contacting said steroid product with alcohol at a temperature of from about 15° C. to about 30° C.

41. A process as set forth in claim 39, wherein the recovered steroid product is washed by contacting said steroid product with alcohol at a temperature of about 20° C.

42. A process as set forth in claim 39, wherein said alcohol is selected from the group consisting of methanol, ethanol, isopropanol, t-butanol and mixtures thereof.

43. A process as set forth in claim 39, wherein said alcohol comprises methanol.

44. A process a set forth in claim 33, wherein the process further comprises drying the recovered steroid product.

45. A process as set forth in claim 44, wherein drying the recovered steroid product comprises contacting the steroid product with air or nitrogen.

46. A process as set forth in claim 45, wherein the steroid product is contacted with nitrogen at a temperature of from about 20° C. to about 80° C.

47. A process as set forth in claim 45, wherein the steroid product is contacted with nitrogen at a temperature of from about 60° C. to about 75° C.

48. A process as set forth in claim 45, wherein the steroid product is contacted with nitrogen at a temperature of about 70° C.

49. A process as set forth in claim 1, wherein the process comprises:
preparing a steroid substrate pre-mixture comprising the steroid substrate and the solvent medium containing the sulfonium salt; and
contacting the base with the steroid substrate pre-mixture.

50. A process as set forth in claim 49, wherein the steroid substrate pre-mixture is prepared by contacting the steroid substrate, the sulfonium salt and a solvent medium.

51. A process as set forth in claim 50, wherein the solvent medium is selected from the group consisting of dimethylsulfoxide, diethyl ether, dioxanes, diglyme, triglyme dimethylformamide, tetrahydrofuran, dimethylacetamide, acetonitrile and mixtures thereof.

52. A process as set forth in claim 49, wherein the base is selected from the group consisting of alkali metal hydroxides, alkali metal hydrides, t-butyl alkali metal alkoxides and alkaline earth metal hydroxides.

53. A process as set forth in claim 52, wherein the base comprises a t-butyl alkali metal alkoxide selected from the group consisting of potassium t-butoxide, sodium t-butoxide, lithium t-butoxide and mixtures thereof.

54. A process as set forth in claim 53, wherein the base comprises potassium t-butoxide.

55. A process as set forth in claim 54, wherein the sulfonium salt comprises a trimethylsulfonium salt.

56. A process as set forth in claim 55, wherein the sulfonium salt comprises trimethylsulfonium methyl sulfate.

57. A process as set forth in claim 54, wherein the solvent medium is selected from the group consisting of dimethylsulfoxide, diethyl ether, dioxanes, diglyme, triglyme dimethylformamide, tetrahydrofuran, dimethylacetamide, acetonitrile and mixtures thereof.

58. A process as set forth in claim 57, wherein the solvent medium comprises tetrahydrofuran.

59. A process as set forth in claim 57, wherein the solvent medium comprises dimethylsulfoxide.

60. A process as set forth in claim 49, wherein the molar ratio of base to sulfonium salt is from about 0.75:1 to about 1.5:1.

61. A process as set forth in claim 49, wherein the molar ratio of base to sulfonium salt is from about 0.9:1 to about 1.1:1.

62. A process as set forth in claim 49, wherein the process further comprises removing solvent from the product mixture by distillation.

63. A process as set forth in claim 49, wherein the process further comprises recovering a steroid product from the product mixture by precipitation, said recovered steroid product comprising the compound of Formula II.

64. A process as set forth in claim 63, wherein said precipitation comprises contacting the product mixture with water.

65. A process as set forth in claim 63, wherein the process further comprises washing the recovered steroid product.

66. A process as set forth in claim 65, wherein the recovered steroid product is washed by contacting said steroid product with water.

67. A process as set forth in claim 66, wherein the recovered steroid product is washed by contacting said steroid product with water at a temperature of at least about 25° C.

68. A process as set forth in claim 66, wherein the recovered steroid product is washed by contacting said steroid product with water at a temperature of at least about 40° C.

69. A process as set forth in claim 66, wherein the recovered steroid product is further washed by contacting said steroid product with an alcohol.

70. A process as set forth in claim 69, wherein the recovered steroid product is washed by contacting said steroid product with alcohol at a temperature of from about 15° C. to about 30° C.

71. A process as set forth in claim 69, wherein the recovered steroid product is washed by contacting said steroid product with alcohol at a temperature of about 20° C.

72. A process as set forth in claim 69, wherein said alcohol is selected from the group consisting of methanol, ethanol, isopropanol, t-butanol and mixtures thereof.

73. A process as set forth in claim 69, wherein said alcohol comprises methanol.

74. A process a set forth in claim 63, wherein the process further comprises drying the recovered steroid product.

75. A process as set forth in claim 74, wherein drying the recovered steroid product comprises contacting the steroid product with air or nitrogen.

76. A process as set forth in claim 74, wherein the steroid product is contacted with nitrogen at a temperature of from about 20° C. to about 80° C.

77. A process as set forth in claim 74, wherein the steroid product is contacted with nitrogen at a temperature of from about 60° C. to about 75° C.

78. A process as set forth in claim 74, wherein the steroid product is contacted with nitrogen at a temperature of about 70° C.

79. A process as set forth in claim 1, wherein the steroid substrate is a compound corresponding to the Formula I-A:

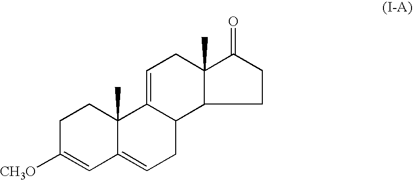

80. A process as set forth in claim 79, wherein the product mixture comprises a compound corresponding to the Formula II-A:

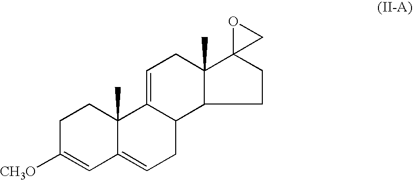

81. A process as set forth in claim 80, wherein the product mixture comprises the β-oriented oxirane compound of Formula II-A in preference to the α-oriented oxirane compound of Formula II-A, said β-oriented oxirane compound of Formula II-A corresponding to the compound of Formula II-C:

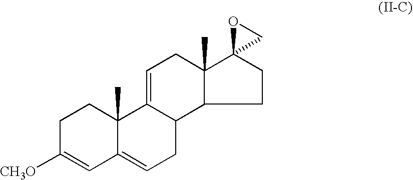

82. A process for the preparation of a steroid compound corresponding to the Formula III: 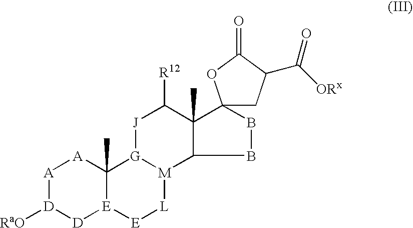
wherein
R^{a} and R^{x} are independently alkyl;
R¹² is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
B-B represents the group —CHR¹⁵—CHR¹⁶— or an α-oriented or β-oriented cyclic group: 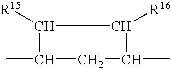
where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
G-J represents the group: 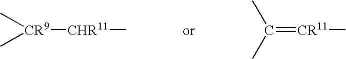
where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
D-D represents the group: 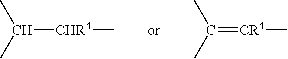
where R⁴ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
E-E represents the group: 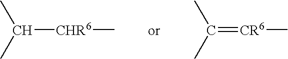
where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and
L-M represents the group: 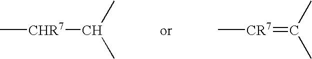
where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl,
the process comprising:
contacting a steroid substrate corresponding to the Formula II: 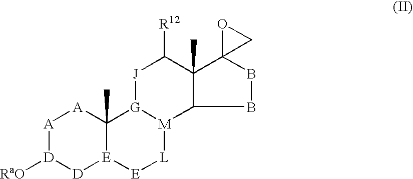
wherein the substituents R^{a}, R^{x}, R¹², A-A, B-B, D-D, E-E, G-J and L-M of the steroid substrate are as defined in Formula III, with a malonic acid diester and a base in the presence of a solvent to produce a product mixture comprising the compound of Formula III; and
treating the product mixture to remove or sequester base.

83. A process as set forth in claim 82, wherein the product mixture comprises a compound of the Formula III-B: 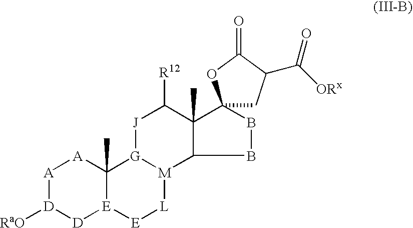
wherein R^{a}, R^{x}, R¹², A-A, B-B, D-D, E-E, G-J and L-M are as defined in Formula III.

84. A process as set forth in claim 82, wherein said treatment of the product mixture comprises removing base from the product mixture.

85. A process as set forth in claim 84, wherein said treatment of the product mixture comprises neutralizing base within the product mixture.

86. A process as set forth in claim 85, wherein the product mixture is treated by contacting said product mixture with an acid.

87. A process as set forth in claim 82, wherein the process comprises:
preparing a steroid substrate pre-mixture comprising the steroid substrate, solvent and the malonic acid diester; and
contacting the base and the steroid substrate pre-mixture.

88. A process as set forth in claim 82 wherein the malonic acid diester comprises an alkyl malonate.

89. A process as set forth in claim 88 wherein the malonic acid diester comprises dimethyl malonate or diethyl malonate.

90. A process as set forth in claim 88, wherein the malonic acid diester comprises diethyl malonate.

91. A process as set forth in claim 82, wherein the base comprises an alkali metal alkoxide.

92. A process as set forth in claim 91 wherein the base comprises sodium methoxide or sodium ethoxide.

93. A process as set forth in claim 82 wherein the malonic acid diester comprises diethyl malonate and the base comprises sodium ethoxide.

94. A process as set forth in claims 82, wherein the solvent is selected from the group consisting of an anhydrous alcohol, dimethylformamide, dimethylsulfoxide, dimethylacetamide and mixtures thereof.

95. A process as set forth in claim 94 wherein the solvent comprises an anhydrous alcohol.

96. A process as set forth in claim 95 wherein the solvent comprises anhydrous ethanol.

97. A process as set forth in claim 82, wherein the product mixture is treated by contact with an acid and said acid is selected to be soluble within the medium of the product mixture.

98. A process as set forth in claim 82, wherein the product mixture is treated by contact with an acid selected from the group consisting of acetic acid, formic acid, propionic acid, sulfuric acid, phosphoric acid and hydrochloric acid.

99. A process as set forth in claim 98, wherein said acid comprises acetic acid.

100. A process as set forth in claim 98, wherein said product mixture is contacted with from about 0.75 to about 1.5 molar equivalents of acid.

101. A process as set forth in claim 98, wherein the product mixture is contacted with about 0.85 to about 1.05 molar equivalents of acid.

102. A process as set forth in claim 82, wherein the process further comprises cooling the product mixture prior to removing or sequestering base within the product mixture.

103. A process as set forth in claim 102, wherein the product mixture is cooled to a temperature of from about 40° to about 75° C. prior to removing or sequestering base within the product mixture.

104. A process as set forth in claim 82, wherein the process further comprises recovering a steroid product from the product mixture, said recovered steroid product comprising the compound of Formula III.

105. A process as set forth in claim 104, wherein the steroid product is recovered from the product mixture by precipitation.

106. A process as set forth in claim 104, wherein the process further comprises washing the recovered steroid product.

107. A process as set forth in claim 106, wherein the recovered steroid product is washed by contacting said steroid product with water.

108. A process as set forth in claim 106, wherein the recovered steroid product is washed by contacting said steroid product with alcohol.

109. A process as set forth in claim 106, wherein the recovered steroid product is washed by contacting said steroid product with a mixture of water and alcohol.

110. A process as set forth in claim 109, wherein said mixture of water and alcohol comprises from about 10% to about 50% by weight alcohol.

111. A process as set forth in claim 109, wherein said mixture of water and alcohol comprises from about 25% to about 35% by weight alcohol.

112. A process as set forth in claim 109, wherein said mixture of alcohol and water comprises about 30% by weight alcohol.

113. A process a set forth in claim 104, wherein the process further comprises drying the recovered steroid product.

114. A process as set forth in claim 113, wherein drying the recovered steroid product comprises contacting the steroid product with air or nitrogen.

115. A process as set forth in claim 113, wherein the steroid product is contacted with nitrogen at a temperature of from about 20° C. to about 70° C.

116. A process as set forth in claim 113, wherein the steroid product is contacted with nitrogen at a temperature of about 60° C.

117. A process as set forth in claim 82, wherein the process comprises:
preparing a pre-mixture comprising the base, the malonic acid diester and the solvent; and
contacting the steroid substrate with said pre-mixture to produce the product mixture.

118. A process as set forth in claim 82 wherein the steroid substrate is a compound corresponding to the Formula II-A:

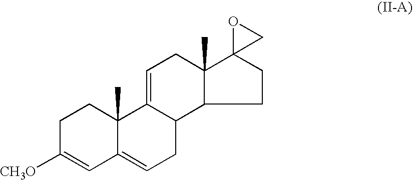

119. A process as set forth in claim 118 wherein the product mixture comprises a compound corresponding to the Formula III-A: 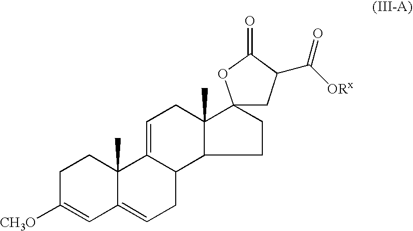
wherein R^{x} is alkyl.

120. A process as set forth in claim 118, wherein the product mixture comprises a compound corresponding to the Formula III-C: 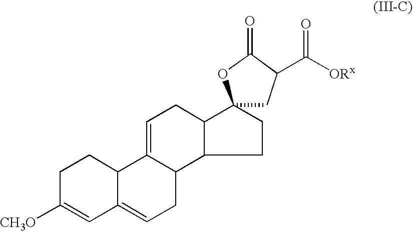
wherein R^{x} is alkyl.

121. A process for the preparation of a steroid compound corresponding to the Formula VI: 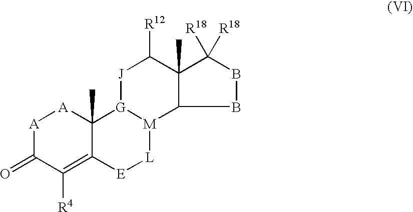
wherein
R⁴ and R¹² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
R¹⁷ and R¹⁸ are independently selected from the group consisting of hydrogen, alkyl, hydroxy, alkenyl and alkynyl or R¹⁷ and R¹⁸ together form a ketal or keto group or R¹⁷ and R¹⁸ together with the C₁₇ carbon to which they are attached form the α-oriented or β-oriented cyclic structure: 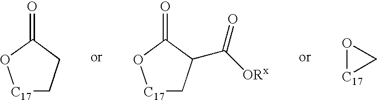
where R^{x} is alkyl;
A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
B-B represents the group —CHR¹⁵—CHR¹⁶— or an α-oriented or β-oriented cyclic group: 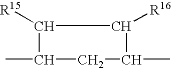
where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
G-J represents the group: 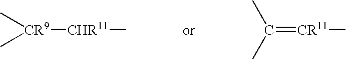
where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
E-L represents the group —CHR⁶—CHR⁷— or —CR⁶═CR⁷—, where R⁶ and R⁷ are independent, R⁶ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy, and R⁷ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl; and
M-G represents the group: 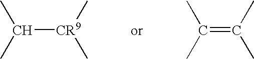
where R⁹ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy,
the process comprising:
oxidizing a steroid substrate corresponding to a compound of the Formula V: 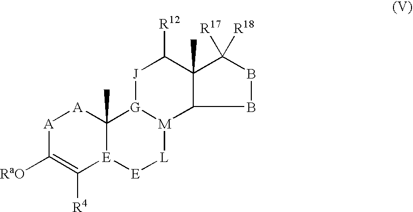
wherein
R^{a} is alkyl;
E-E represents the group: 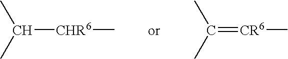
where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
L-M represents the group: 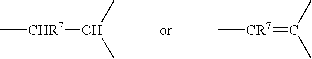
where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl, and
the substituents R⁴, R¹², R¹⁷, R¹⁸, A-A, B-B, and G-J are as defined in Formula VI.

122. A process as set forth in claim 121, wherein the steroid substrate corresponds to a compound of Formula V-A: 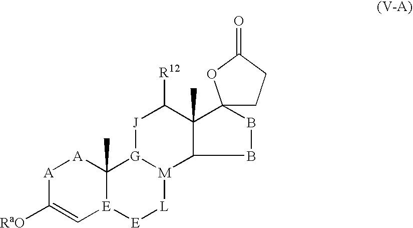
wherein the substituents R^{a}, E-E and L-M are as defined in Formula V and the substituents R⁴, R¹², A-A, B-B and G-J are as defined in Formula VI.

123. A process as set forth in claim 122, wherein the product mixture comprises a steroid compound corresponding to a compound of Formula VI-A: 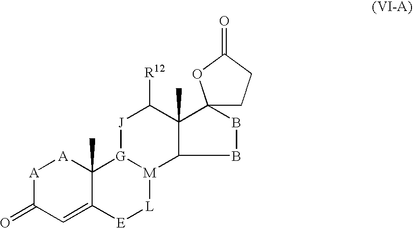
wherein the substituents R⁴, R¹², A-A, B-B, G-J, E-L and M-G are as defined in Formula VI.

124. A process as set forth in claim 122, wherein the product mixture comprises a steroid compound corresponding to a compound of Formula VI-B: 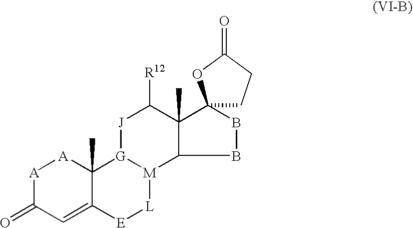
wherein the substituents R⁴, R¹², A-A, B-B, G-J, E-L and M-G are as defined in Formula VI.

125. A process as set forth in claim 121, wherein the process comprises contacting the steroid substrate of Formula V with an oxidizing agent in the presence of water to produce a product mixture comprising the steroid compound of Formula VI.

126. A process as set forth in claim 125, wherein the oxidizing agent is selected from the group consisting of o-chloranil, p-chloranil, dichlorodicyanobenzoquinone and mixtures thereof.

127. A process as set forth in claim 125, wherein the oxidizing agent comprises p-chloranil.

128. A process as set forth in claim 125, wherein the steroid substrate is contacted with an amount of oxidizing agent which is in excess of the stoichiometric amount of oxidizing agent required for the oxidation of the steroid substrate.

129. A process as set forth in claim 128, wherein the steroid substrate is contacted with about 1.01 to about 1.50 molar equivalents of oxidizing agent.

130. A process as set forth in claim 128, wherein the steroid substrate is contacted with about 1.01 to about 1.25 molar equivalents of oxidizing agent.

131. A process as set forth in claim 128, wherein the steroid substrate is contacted with about 1.01 to about 1.05 molar equivalents of oxidizing agent.

132. A process as set forth in claim 125, wherein the steroid substrate and the oxidizing agent are contacted in the presence of a solvent.

133. A process as set forth in claim 132, wherein the process comprises:
introducing the steroid substrate and the oxidizing agent into a reaction zone; and
thereafter contacting said steroid substrate and said oxidizing agent in said reaction zone with said solvent and water.

134. A process as set forth in claim 132, wherein the process comprises:
preparing a substrate pre-mixture comprising the steroid substrate and the oxidizing agent; and
contacting the substrate pre-mixture with said solvent and water.

135. A process as set forth in claim 132, wherein the process comprises:
contacting the steroid substrate and the oxidizing agent with a premixed reaction medium comprising said solvent and water.

136. A process as set forth in claim 132, wherein the solvent is selected from the group consisting of dimethylformamide, acetonitrile, methanol, acetone, methylene chloride and mixtures thereof.

137. A process as set forth in claim 132, wherein the solvent comprises methylene chloride.

138. A process as set forth in claim 132, wherein the solvent comprises a mixture of methylene chloride and methanol.

139. A process as set forth in claim 132, wherein the solvent and water are mixed prior to contacting the steroid substrate and the oxidizing agent.

140. A process as set forth in claim 125, wherein the process further comprises isolating the steroid compound of Formula VI from the product mixture.

141. A process as set forth in claim 125, wherein the process further comprises contacting the product mixture with a reducing agent.

142. A process as set forth in claim 141, wherein the reducing agent is selected from the group consisting of sulfite, metabisulfite, and mixtures thereof.

143. A process as set forth in claim 125, wherein the product mixture further comprises a substituted dihydroquinone byproduct.

144. A process as set forth in claim 143 wherein the process further comprises removing the substituted dihydroquinone byproduct from the product mixture and recovering the steroid compound of Formula VI.

145. A process as set forth in claim 144 wherein removing said substituted dihydroquinone by-product from the product mixture comprises contacting the product mixture with a base.

146. A process as set forth in claim 145 wherein said product mixture is contacted with a base under essentially anhydrous conditions.

147. A process as set forth in claim 145, wherein the base comprises an alkali metal hydroxide selected from the group consisting of NaOH, LiOH, KOH, and mixtures thereof.

148. A process as set forth in claim 147, wherein the base comprises a solid particulate.

149. A process as set forth in claim 148, wherein the base comprises potassium hydroxide.

150. A process as set forth in claim 121, wherein the oxidation process comprises
contacting the steroid substrate with a source of a halogen in the presence of water to produce a halogenated steroid intermediate; and
dehydrohalogenating the halogenated steroid intermediate with a base to produce a product mixture comprising the steroid product of Formula VI.

151. A process as set forth in claim 121, wherein the steroid substrate is selected from the group consisting of 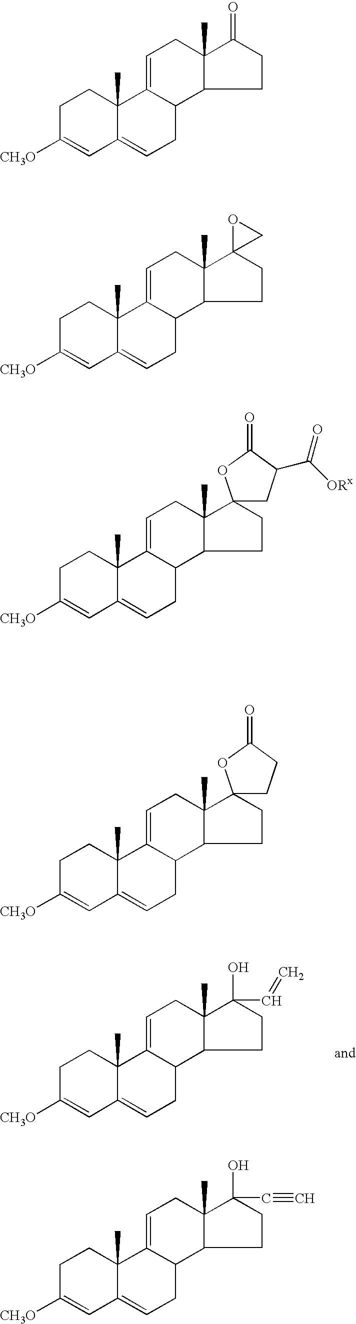
wherein R^{x} is alkyl.

152. A process as set forth in claim 121, wherein the steroid substrate is selected from the group consisting of 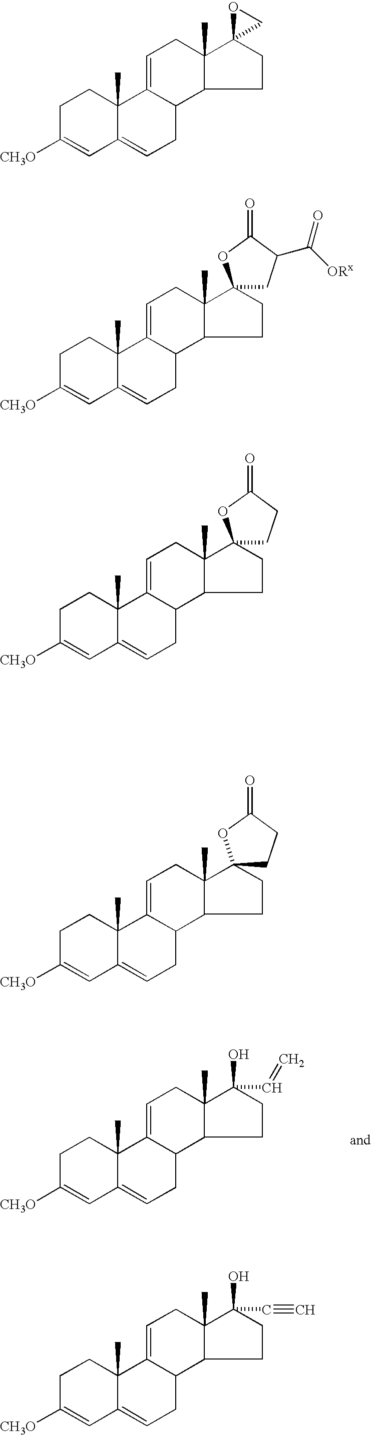
wherein R^{x} is alkyl.

152. A process as set forth in claim 121, wherein the steroid product of Formula VI is selected from the group consisting of 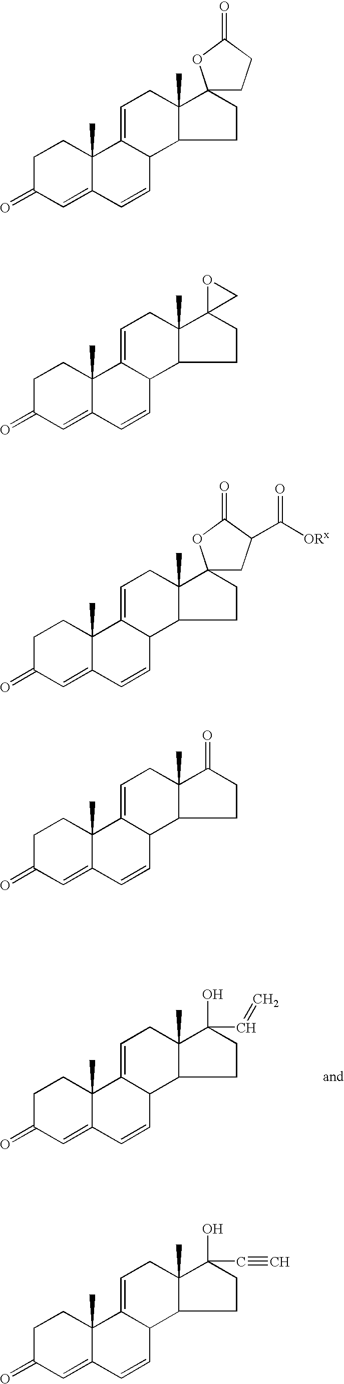
wherein R^{x} is alkyl.

154. A process as set forth in claim 121, wherein the steroid product of Formula VI is selected from the group consisting of 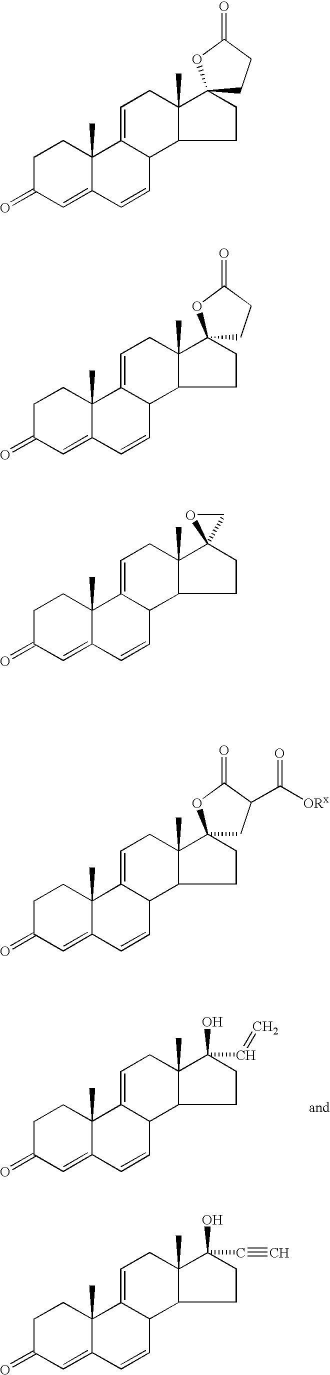
where R^{x} is alkyl.

155. A process for the preparation of a steroid compound corresponding to the Formula VI: 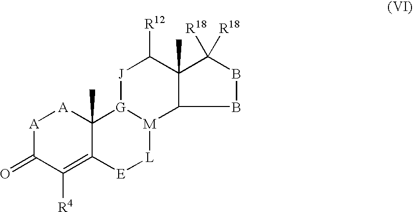
wherein
R⁴ and R¹² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
R¹⁷ and R¹⁸ are independently selected from the group consisting of hydrogen, alkyl, hydroxy, alkenyl and alkynyl or R¹⁷ and R¹⁸ together form a ketal or keto group or R¹⁷ and R¹⁸ together with the C₁₇ carbon to which they are attached form the α-oriented or β-oriented cyclic structure: 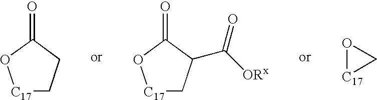
where R^{x} is alkyl;
A-A represents the group —CHR¹—CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
B-B represents the group —CHR¹⁵—CHR¹⁶— or an α-oriented or β-oriented cyclic group: 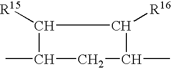
where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
G-J represents the group: 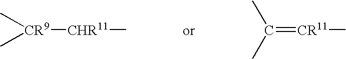
where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
E-L represents the group —CHR⁶—CHR⁷— or —CR⁶═CR⁷—, where R⁶ and R⁷ are independent, R⁶ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy, and R⁷ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl; and
M-G represents the group: 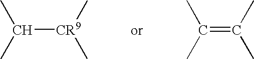
where R⁹ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy,
the process comprising:
contacting a steroid substrate corresponding to a compound of the Formula V: 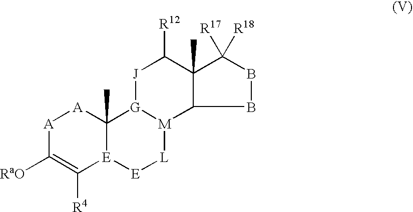
wherein
R^{a} is alkyl;
E-E represents the group: 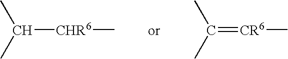
where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
L-M represents the group: 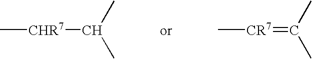
where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl, and
the substituents R⁴, R¹², R¹⁷, R¹⁸, A-A, B-B, and G-J are as defined in Formula VI,
with an oxidizing agent in the presence of water to produce a product mixture comprising the steroid compound of Formula VI; and
contacting the product mixture with a base.

156. A process as set forth in claim 155, wherein the product mixture is contacted with a base under essentially anhydrous conditions.

157. A process as set forth in claim 155, wherein the base comprises an alkali metal hydroxide selected from the group consisting of NaOH, LiOH, KOH, and mixtures thereof.

158. A process as set forth in claim 157, wherein the base comprises a solid particulate.

159. A process as set forth in claim 158, wherein the base comprises potassium hydroxide.

160. A process as set forth in claim 155, wherein the process further comprises contacting the product mixture with a reducing agent prior to contacting the product mixture with a base.

161. A process as set forth in claim 160, wherein the reducing agent is selected from the group consisting of sulfite, metabisulfite, and mixtures thereof.

162. A process as set forth in claim 155, wherein the process further comprises recovering the steroid compound of Formula VI from the product mixture.

163. A process as set forth in claim 162, wherein the steroid compound of Formula VI is recovered from the product mixture by precipitation.

164. A process as set forth in claim 155, wherein the oxidizing agent is selected from the group consisting of o-chloranil, p-chloranil, dichlorodicyanobenzoquinone and mixtures thereof.

165. A process as set forth in claim 155, wherein the oxidizing agent comprises p-chloranil.

166. A process as set forth in claim 155, wherein the steroid substrate is contacted with an amount of oxidizing agent which is in excess of the stoichiometric amount of oxidizing agent required for oxidizing the steroid substrate.

167. A process as set forth in claim 166, wherein the steroid substrate is contacted with about 1.01 to about 1.50 molar equivalents of oxidizing agent.

168. A process as set forth in claim 166, wherein the steroid substrate is contacted with about 1.01 to about 1.25 molar equivalents of oxidizing agent.

169. A process as set forth in claim 166, wherein the steroid substrate is contacted with about 1.01 to about 1.05 molar equivalents of oxidizing agent.

170. A process as set forth in claim 155, wherein the steroid substrate and the oxidizing agent are contacted in the presence of a solvent.

171. A process as set forth in claim 170, wherein the process comprises:
introducing the steroid substrate and the oxidizing agent into a reaction zone; and
thereafter contacting said steroid substrate and said oxidizing agent in said reaction zone with said solvent and water.

172. A process as set forth in claim 170, wherein the process comprises:
preparing a substrate pre-mixture comprising the steroid substrate and the oxidizing agent; and
contacting the substrate pre-mixture with said solvent and water.

173. A process as set forth in claim 170, wherein the process comprises:
contacting the steroid substrate and the oxidizing agent with a pre-mixed reaction medium comprising said solvent and water.

174. A process as set forth in claim 170, wherein the solvent is selected from the group consisting of dimethylformamide, acetonitrile, methanol, acetone, methylene chloride and mixtures thereof.

175. A process as set forth in claim 170, wherein the solvent comprises methylene chloride.

176. A process as set forth in claim 170, wherein the solvent comprises a mixture of methylene chloride and methanol.

177. A process as set forth in claim 170, wherein the solvent and water are mixed prior to contacting the steroid substrate and the oxidizing agent.

178. A process as set forth in claim 155, wherein the steroid substrate corresponds to a compound of Formula V-A: 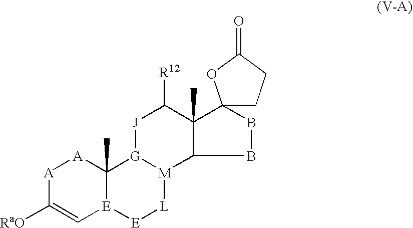
wherein the substituents R^{a}, E-E and L-M are as defined in Formula V and the substituents R⁴, R¹², A-A, B-B and G-J are as defined in Formula VI.

179. A process as set forth in claim 178, wherein the product mixture comprises a steroid compound corresponding to a compound of Formula VI-A: 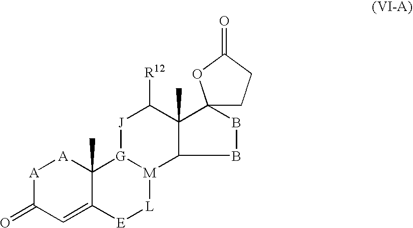
wherein the substituents R⁴, R¹², A-A, B-B, G-J, E-L and M-G are as defined in Formula VI.

180. A process as set forth in claim 178, wherein the product mixture comprises a steroid compound corresponding to a compound of Formula VI-B: 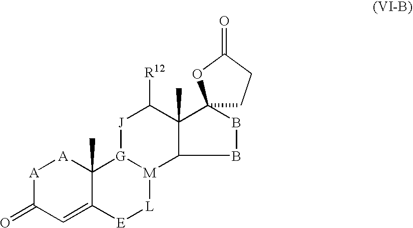
wherein the substituents R⁴, R¹², A-A, B-B, G-J, E-L and M-G are as defined in Formula VI.

181. A process as set forth in claim 155, wherein the steroid substrate is selected from the group consisting of 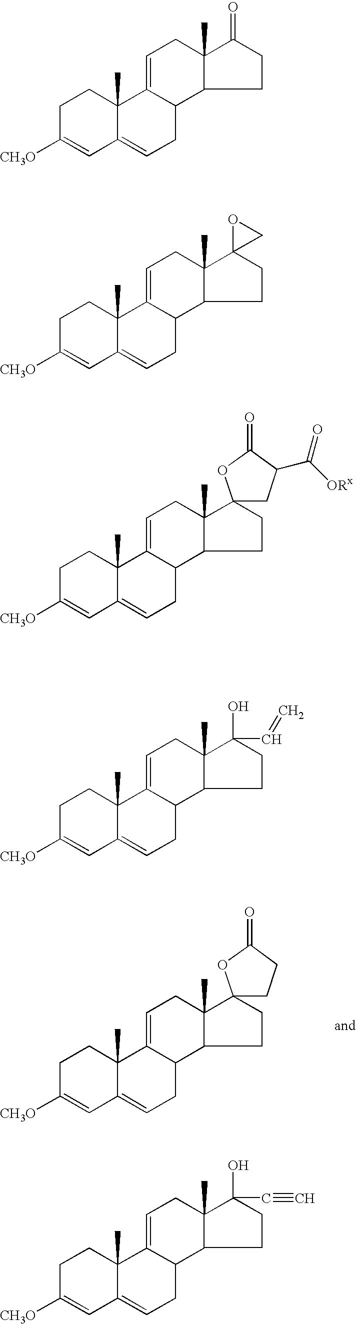
wherein R^{x} is alkyl.

182. A process as set forth in claim 155, wherein the steroid substrate is selected from the group consisting of 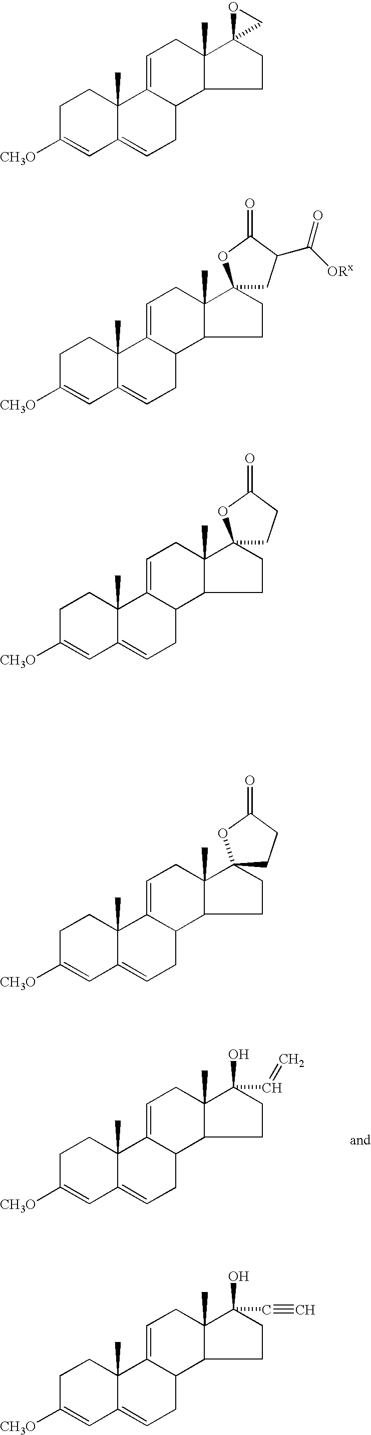
wherein R^{x} is alkyl.

183. A process as set forth in claim 155, wherein the steroid product of Formula VI is selected from the group consisting of: 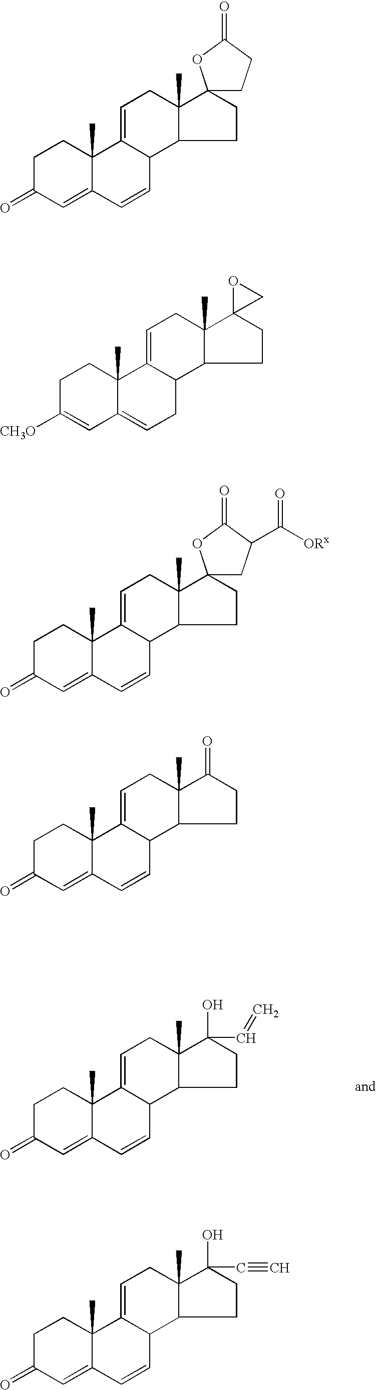
wherein R^{x} is alkyl.

184. A process as set forth in claim 155, wherein the steroid product of Formula VI is selected from the group consisting of 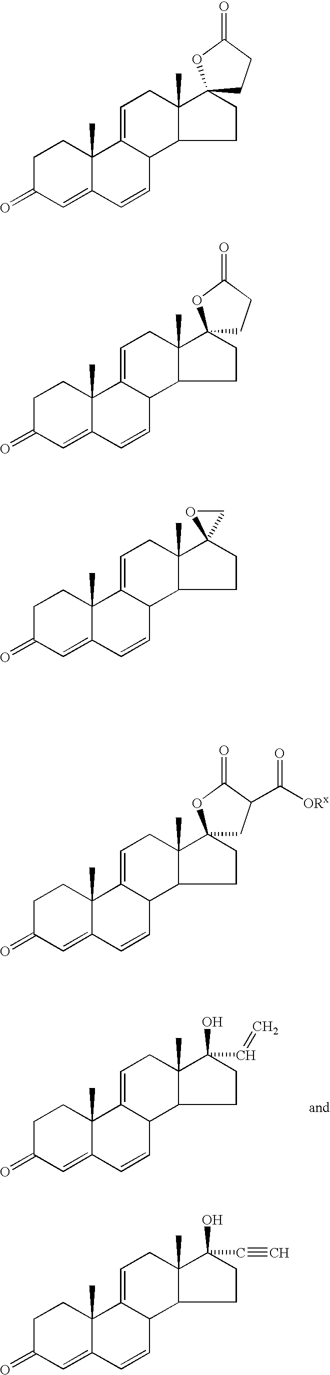
wherein R^{x} is alkyl.

185. A process for the preparation of a steroid compound corresponding to Formula VI-A: 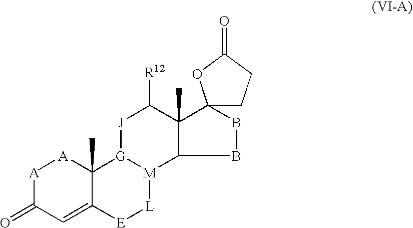
wherein:
R¹² is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
A-A represents the group —CHR¹-CHR²— or —CR¹═CR²—, where R¹ and R² are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
B-B represents the group —CHR¹⁵—CHR¹⁶— or an α-oriented or β-oriented cyclic group: 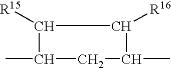
where R¹⁵ and R¹⁶ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
G-J represents the group: 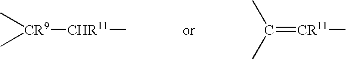
where R⁹ and R¹¹ are independently selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
E-L represents the group —CHR⁶—CHR⁷— or —CR⁶═CR⁷—, where R⁶ and R⁷ are independent, R⁶ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy; and R⁷ being selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl; and
M-G represents the group: 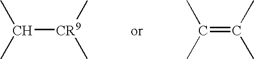
where R⁹ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy,
the process comprising:
contacting a steroid substrate corresponding to a compound of Formula I: 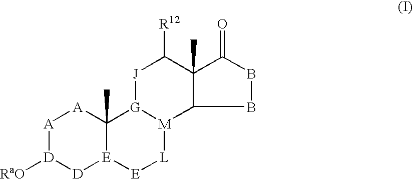
wherein:
R^{a} is alkyl;
D-D represents the group: 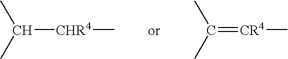
where R⁴ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
E-E represents the group: 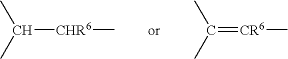
where R⁶ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl and aryloxy;
L-M represents the group: 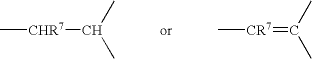
where R⁷ is selected from the group consisting of hydrogen, halo, hydroxy, alkyl, alkoxy, acyl, hydroxyalkyl, alkoxyalkyl, hydroxycarbonyl, alkoxycarbonyl, acyloxyalkyl, cyano, nitro, thioalkyl, aryl, aryloxy, heteroaryl, heterocyclyl, furyl and substituted furyl; and
the substituents R¹², A-A, B-B and G-J are as defined in Formula VI-A,
with a base and a solvent medium containing a sulfonium salt to produce an oxirane intermediate steroid compound of Formula II: 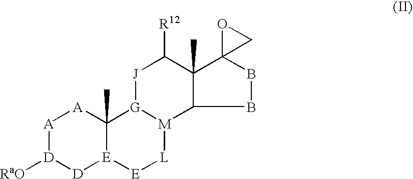
wherein the substituents R^{a}, R¹², A-A, B-B, G-J, D-D, E-E and L-M are as defined in Formula I;
contacting the oxirane intermediate compound of Formula II with a malonic acid diester and a base in the presence of a solvent to produce a dicarboxylate intermediate steroid compound corresponding to Formula III: 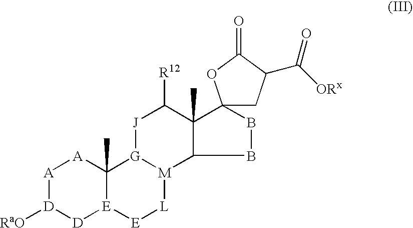
wherein Rx is alkyl and the substituents R^{a}, R¹², A-A, B-B, G-J, D-D, E-E and L-M are as defined in Formula I;
decarboxylating the dicarboxylate intermediate compound of Formula III to produce an enol ether steroid compound of Formula V-A: 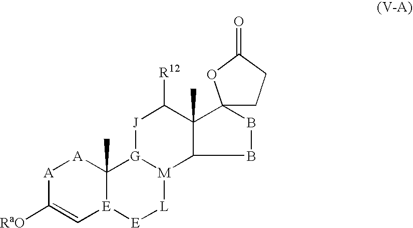
wherein the substituents R^{a}, R¹², A-A, B-B, G-J, E-E and L-M are as defined in Formula I; and
oxidizing the enol ether steroid compound of Formula V-A to produce the steroid compound of Formula VI-A.

186. A process for the preparation of a steroid compound corresponding to Formula VI-C: 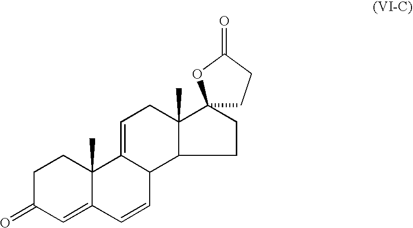
the process comprising:
contacting a steroid substrate corresponding to Formula I-A: 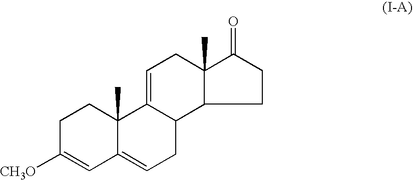
with a base and a solvent medium containing a sulfonium salt to produce an oxirane intermediate compound of Formula II-C: 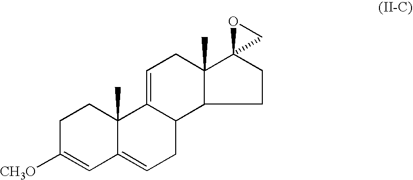
contacting the oxirane intermediate compound of Formula II-C with a malonic acid diester and a base in the presence of a solvent to produce a dicarboxylate intermediate steroid compound corresponding to Formula III-C: 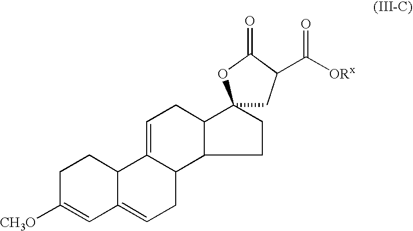
decarboxylating the dicarboxylate intermediate compound of Formula III-C to produce an enol ether steroid compound of Formula IV-C: 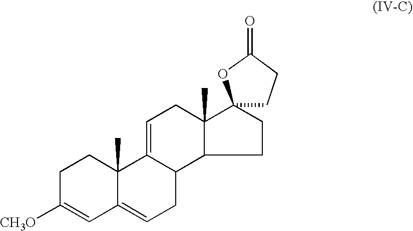
and
oxidizing the enol ether steroid compound of Formula IV-C to produce the steroid compound of Formula VI-C.

187. A process for the preparation of a compound corresponding to Formula X: 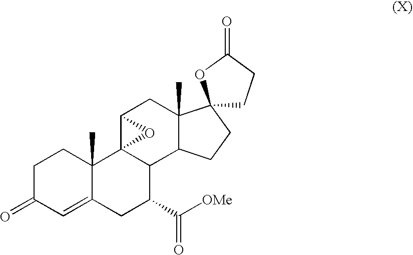
the process comprising:
contacting a steroid substrate of Formula I-A: 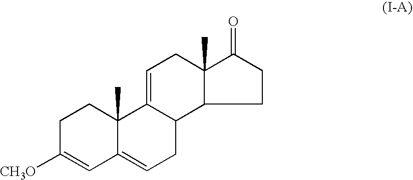
with a base and a solvent medium containing a sulfonium salt to produce an oxirane intermediate steroid compound of Formula II-C: 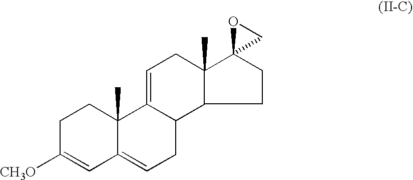
contacting the oxirane intermediate steroid compound of Formula II-C with a malonic acid diester and a base in the presence of a solvent to produce a dicarboxylate intermediate steroid compound of Formula III-C: 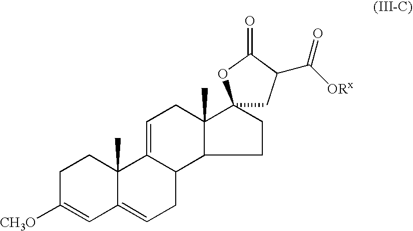
wherein R^{x} is alkyl;
decarboxylating the dicarboxylate intermediate steroid compound of Formula III-C to produce an enol ether steroid compound of Formula IV-C: 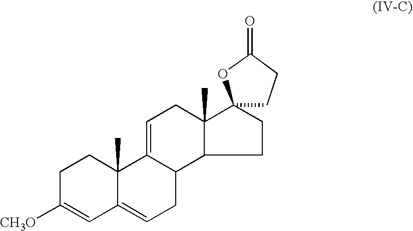
oxidizing the enol ether steroid compound of Formula IV-C to produce a dienone steroid compound of Formula VI-C: 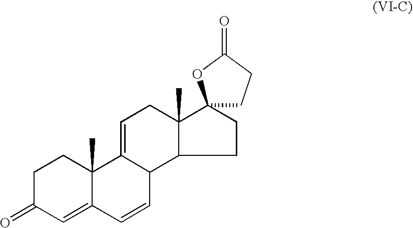
contacting the dienone steroid compound of Formula VI-C with an alkyl furan and a Lewis acid to produce a 7α-furyl intermediate steroid compound of Formula VII: 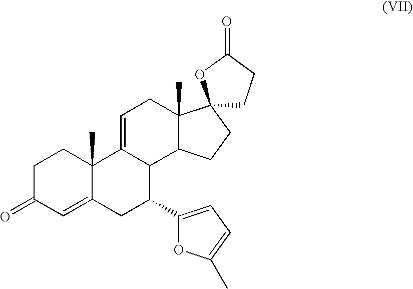
preparing a 7α-methoxycarbonyl intermediate steroid compound of Formula IX from the 7α-furyl intermediate steroid compound of Formula VII, said compound of Formula IX comprising: 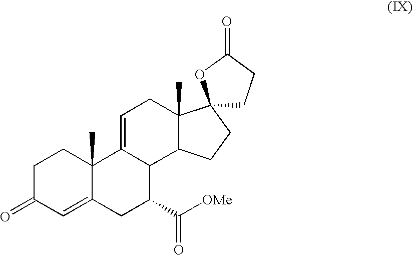
and;
converting the 7α-methoxycarbonyl intermediate steroid compound of Formula IX to the steroid compound of Formula X.

188. A steroid compound corresponding to Formula X: 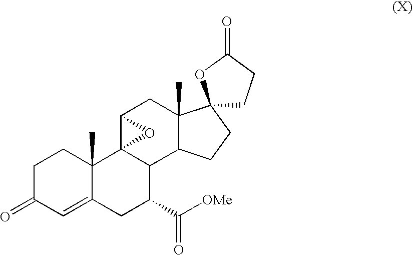
prepared by a process comprising:
contacting a steroid substrate of Formula I-A: 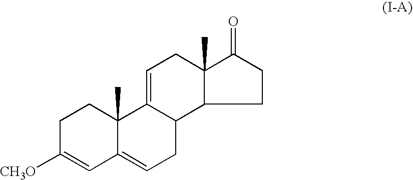
with a base and a solvent medium containing a sulfonium salt to produce an oxirane intermediate steroid compound of Formula II-C: 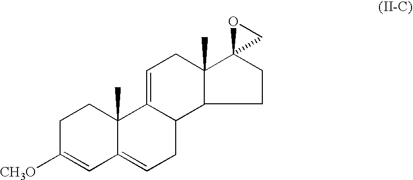
contacting the oxirane intermediate steroid compound of Formula II-C with a malonic acid diester and a base in the presence of a solvent to produce a dicarboxylate steroid compound of Formula III-C: 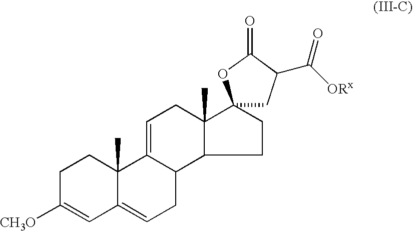
wherein R^{x} is alkyl;
decarboxylating the dicarboxylate steroid compound of Formula III-C to produce an enol ether steroid compound of Formula IV-C: 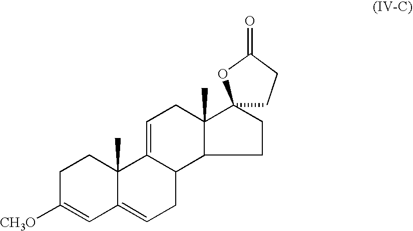
oxidizing the enol ether steroid compound of Formula IV-C to produce a dienone steroid compound of Formula VI-C: 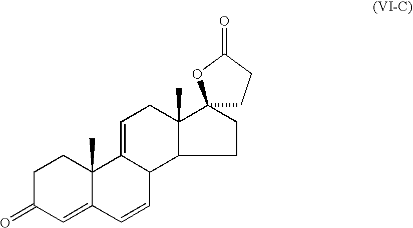
contacting the dienone steroid compound of Formula VI-A with an alkyl furan and a Lewis acid to produce a 7α-furyl intermediate steroid compound of Formula VII: 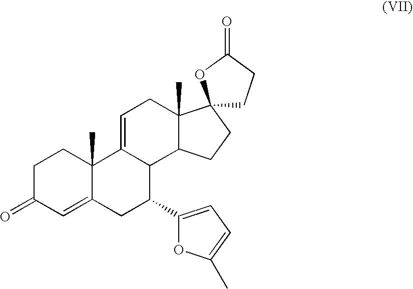
preparing a 7α-methoxycarbonyl intermediate steroid compound of Formula IX from the 7α-furyl intermediate steroid compound of Formula VII, said compound of Formula IX comprising: 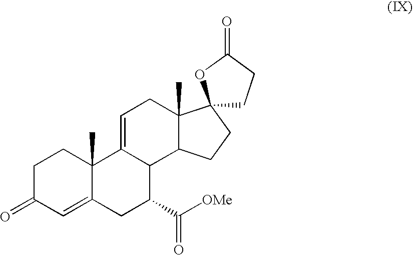
and;
converting the 7α-methoxycarbonyl intermediate steroid compound of Formula IX to the steroid compound of Formula X.
